(19) United States
(12) Patent Application Publication
(10) Pub. No.: US 2024/0287051 A1
(43) Pub. Date: Aug. 29, 2024

(54) PROCESS FOR PREPARING AN ERK INHIBITOR

(71) Applicant: Otsuka Pharmaceutical Co., Ltd., Tokyo (JP)

(72) Inventors: Prabhubas BODHURI, Mountain House, CA (US); Hem Raj KHATRI, Indianapolis, IN (US); Ian SCOTT, Niwot, CO (US); Michael READER, Cambridge (GB); David Charles LATHBURY, Loughborough (GB); Nipun DAVAR, Pleasanton, CA (US); Matthew JOHNSON, Raleigh, NC (US)

(21) Appl. No.: 18/568,772

(22) PCT Filed: Jun. 10, 2022
(86) PCT No.: PCT/IB2022/055416

§ 371 Date: Dec. 8, 2023

Related U.S. Application Data
(60) Provisional application No. 63/209,877, filed on Jun. 11, 2021.
(60) Provisional application No. 63/273,326, filed on Oct. 29, 2021.

(51) Int. Cl. C07D 405/14; C07C 67/10; C07C 217/70; C07C 271/16; C07C 313/06; C07D 209/46; C07D 405/12; C07F 5/02; C12N 9/10

(52) U.S. Cl. CPC C07D 405/14; C07C 67/10; C07C 217/70; C07C 271/16; C07C 313/06; C07D 209/46; C07D 405/12; C07F 5/025; C12N 9/1096; C12Y 206/01029

(57) ABSTRACT

The present disclosure relates generally to methods for the preparation of Compound (I).

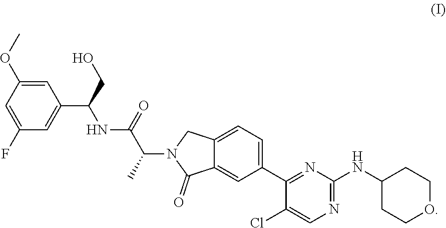 [SMILES: COc1cc(F)cc([C@@H](CO)NC(=O)[C@@H](C)N2Cc3ccc(-c4nc(NC5CCOCC5)ncc4Cl)cc3C2=O)c1]

### CROSS REFERENCE TO RELATED APPLICATIONS

This application claims the benefit under 35 U.S.C. § 119(e) of U.S. Provisional Application No. 63/209,877 filed Jun. 11, 2021, and U.S. Provisional Application No. 63/273,326 filed Oct. 29, 2021, which are incorporated herein by reference in their entirety.

### FIELD

The present application relates to processes for synthesis of an ERK1/2 inhibitor, novel intermediates, and methods for synthesizing the same.

### BACKGROUND

The extracellular signal regulated kinases (ERK 1/2) are ubiquitously expressed protein serine/threonine kinases that comprise a key component of the mitogen-activated protein kinase (MAPK) signaling pathway. The MAPK pathway is an evolutionary conserved cell signaling pathway that regulates a variety of cellular processes including cell cycle progression, cell migration, cell survival, differentiation, metabolism, proliferation and transcription. ERK1/2 activity is commonly upregulated in cancer, as a result of activating mutations within upstream components of the MAPK pathway. ERK1/2 inhibitors are useful in therapy, in particular in the treatment of cancer.

Improved methods for synthesizing ERK1/2 inhibitor compounds are disclosed herein.

### SUMMARY

The present disclosure, in one embodiment, provides methods for the synthesis of a compound of Formula (I) (“Compound (I)”), or a pharmaceutically acceptable salt, solvate, or hydrate thereof:

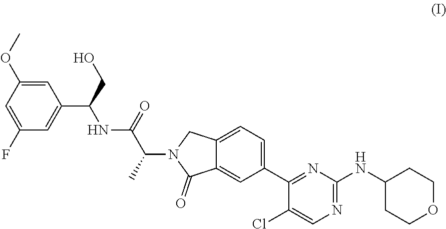 [SMILES: COc1cc(F)cc([C@@H](CO)NC(=O)[C@@H](C)N2Cc3ccc(-c4nc(NC5CCOCC5)ncc4Cl)cc3C2=O)c1]

comprising the use of intermediates of Formula (C), Formula (D), Formula (G-1), Formula (G-3), Formula (H-1), Formula (J-3), Formula (J-4), Formula (S), and/or other intermediates described herein. Also provided are improved methods for the synthesis of an intermediate of Formula (J-2) and the use thereof for the synthesis of Compound (I). Compound (I) is named ((2R)-2-(6-{5-chloro-2-[(tetrahydro-2H-pyran-4-yl)amino]pyrimidin-4-yl}-1-oxo-2,3-dihydro-1H-isoindol-2-yl)-N-[(S)-1-(3-fluoro-5-methoxyphenyl)-2-hydroxyethyl]propanamide.

In one aspect, provided herein is a method for the preparation of a compound of Formula (D) or a salt thereof

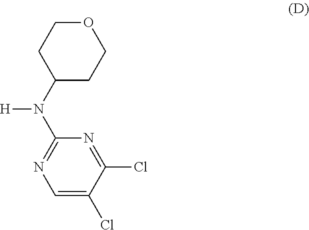 [SMILES: Clc1cnc(NC2CCOCC2)nc1Cl]

as described in the detailed description and the Examples sections.

In one aspect, provided herein is a method for the preparation of a compound of Formula (J) or a stereoisomer thereof, or a salt thereof,

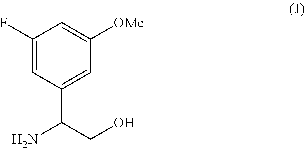 [SMILES: COc1cc(F)cc(C(N)CO)c1]

as described in the detailed description and the Examples sections.

In one aspect, provided herein is a compound of Formula (G-1) or a salt thereof

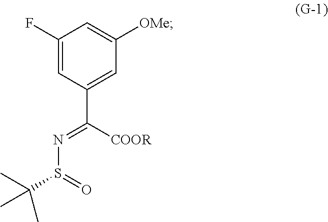

wherein R is C₁₋₅ alkyl.

In one aspect, provided herein is a compound of Formula (G-2) or a stereoisomer thereof, or a salt thereof

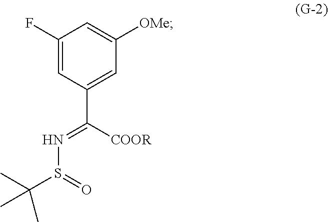

wherein R is C₁₋₅ alkyl.

In one aspect, provided herein is a compound of Formula (G-3) or a salt thereof

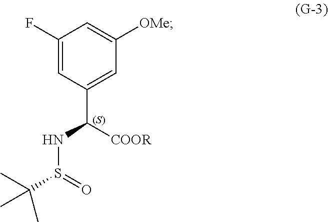

wherein R is C₁₋₅ alkyl.

In one aspect, provided herein is a compound of Formula (H-1), or a salt thereof,

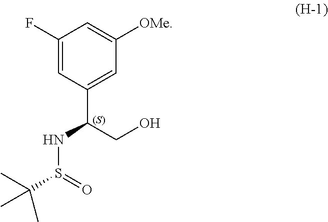

In one aspect, provided herein is a compound of Formula (J-3)

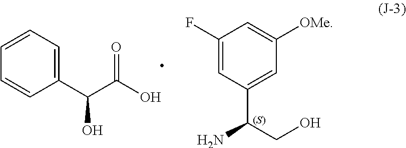

In one aspect, provided herein is a compound of Formula (J-4)

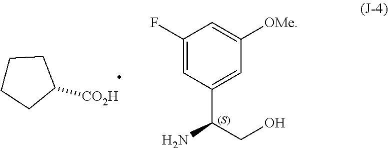

In one aspect, provided herein is a compound of Formula (M-2), or a salt thereof,

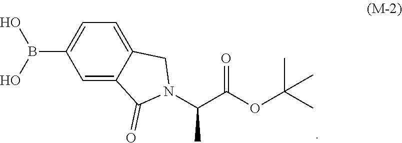 [SMILES: C[C@H](C(=O)OC(C)(C)C)N1Cc2ccc(B(O)O)cc2C1=O]

In one aspect, provided herein is a compound of Formula (S), or a salt thereof,

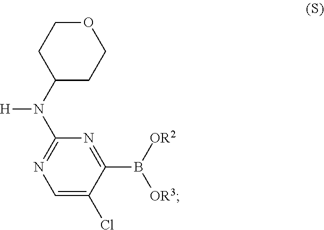

wherein R² and R³ are independently H, C₁₋₅ alkyl, or R² and R³ together with the atoms to which they are attached form a 5-or 6-membered ring optionally substituted with 1, 2, 3, or 4 C₁₋₃ alkyl.

In one aspect, provided herein is a method for the preparation of a compound of Formula (N), or a salt thereof,

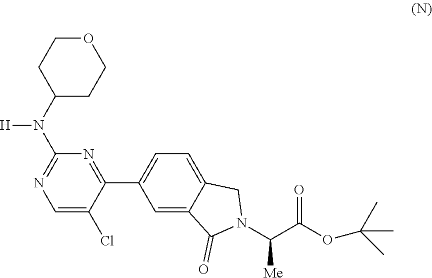 [SMILES: C[C@H](C(=O)OC(C)(C)C)N1Cc2ccc(-c3nc(NC4CCOCC4)ncc3Cl)cc2C1=O]

as described in the detailed description and the Examples sections.

### BRIEF DESCRIPTION OF DRAWINGS

FIG. 1 shows an X-ray powder diffraction pattern for Form B of Compound (I) prepared by the methods described herein.

FIG. 2 shows a single crystal X-ray structure for Form B of Compound (I) as an ORTEP plot.

### DETAILED DESCRIPTION

Compound (I) has been described as Example 685 in WO 2017/068412, which reference is incorporated herein by reference in its entirety. Compound (I) is useful for the treatment of cancer and other conditions described in WO 2017/068412.

The methods described in WO 2017/068412 for the synthesis of Compound (I) comprise the reaction of trichloropyrimidine (X) with a boronate compound (M-1) with a subsequent reaction with a tetrahydropyranyl amine (B) as shown in Scheme A below.

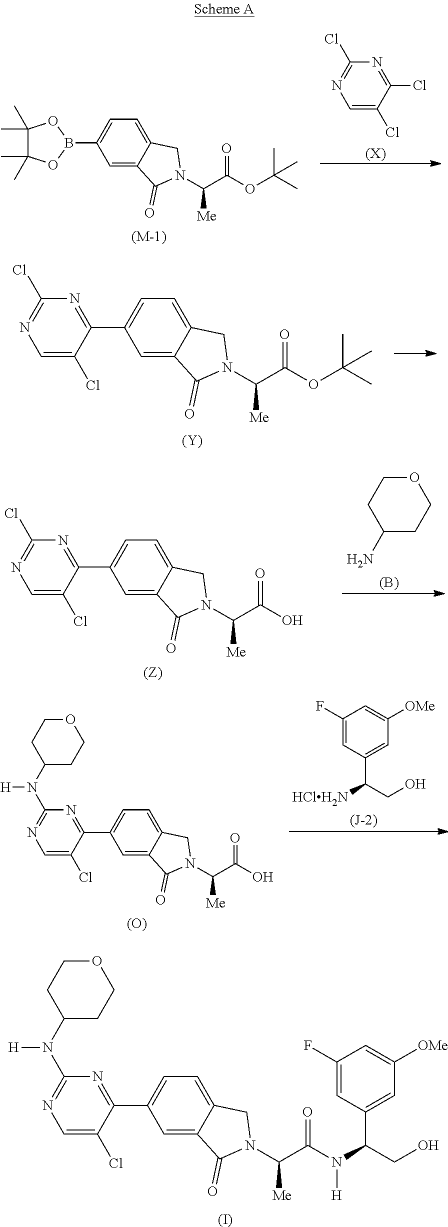

It was determined that the reaction of (M-1)) with (X) to provide (Y) leads to undesirable side reactions which is problematic during large scale manufacturing. The Suzuki coupling reaction between compound (M-1) and (X) leads to formation of compounds other than (Y) due to the presence of multiple chlorine atoms in compound (X). Typically, the Suzuki coupling between (M-1) and (X) provided about 50% yield (35.9% area by HPLC) of the bis-coupled impurity and about 33.4% area by HPLC of (Y) and additional impurities associated with hydrolysis of (X). Further, the reaction of (Z) with (B) requires harsh conditions including the use of N-methyl pyrrolidine (NMP) as a solvent, can only be done on small scale, and leads to telescoping of two impurities, (Z-1) and (Z-2), shown below, that are difficult to separate from the final compound (I).

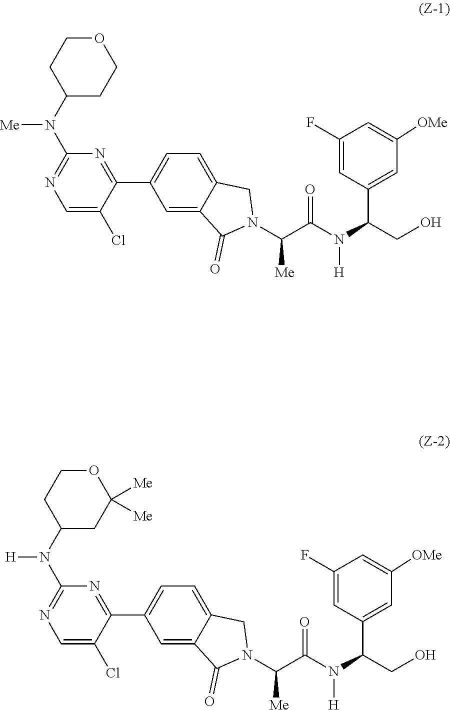 [SMILES: COc1cc(F)cc([C@@H](CO)NC(=O)[C@@H](C)N2Cc3ccc(-c4nc(N(C)C5CCOCC5)ncc4Cl)cc3C2=O)c1 ; COc1cc(F)cc([C@@H](CO)NC(=O)[C@@H](C)N2Cc3ccc(-c4nc(NC5CCOC(C)(C)C5)ncc4Cl)cc3C2=O)c1]

Described herein is a method for the preparation of a compound of Formula (D) and the use of Compound (D) for the preparation of Compound (N). Advantageously, the use of Compound (D) avoids the formation of undesirable side products and provides higher yields of Compound (N) thereby providing an improved process for the preparation of Compound (I).

A further improvement described herein is the preparation of a compound of Formula (J-2) having improved purity and improved chiral purity, as described herein in Scheme 1 and in the Examples, and the use thereof for the preparation of Compound (I).

### Definitions

The following description sets forth exemplary embodiments of the present technology. It should be recognized, however, that such description is not intended as a limitation on the scope of the present disclosure but is instead provided as a description of exemplary embodiments.

As used in the present specification. the following words, phrases and symbols are generally intended to have the meanings as set forth below, except to the extent that the context in which they are used indicates otherwise.

As used herein, a “compound of Formula (XX)” is used interchangeably with “Formula XX”, “Compound (XX)”. “Compound XX”. “XX” or “(XX)”.

A dash (“—”) that is not between two letters or symbols is used to indicate a point of attachment for a substituent. For example, —C(O)NH₂ is attached through the carbon atom. A dash at the front or end of a chemical group is a matter of convenience: chemical groups may be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named.

The prefix “Cᵤ₋ᵥ” indicates that the following group has from u to v carbon atoms. For example, “C₁₋₆ alkyl” indicates that the alkyl group has from 1 to 6 carbon atoms.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±1%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

“Alkyl” refers to an unbranched or branched saturated hydrocarbon chain. As used herein, alkyl has 1 to 20 carbon atoms (i.e., C₁₋₂₀ alkyl), 1 to 8 carbon atoms (i.e., C₁₋₈ alkyl), 1 to 6 carbon atoms (i.e., C₁₋₆ alkyl), or 1 to 4 carbon atoms (i.e., C₁₋₄ alkyl). Examples of alkyl groups include methyl, ethyl, propxl, isopropyl, n-butyl, sec-butyl, iso-butyl, tert-butyl, pentyl, 2-pentyl, isopentyl, neopentyl, hexyl, 2-hexyl, 3-hexyl, and 3-methylpentyl. When an alkyl residue having a specific number of carbons is named by chemical name or identified by molecular formula, all positional isomers having that number of carbons may be encompassed; thus, for example, “butyl” includes n-butyl (i.e. —(CH₂)₃CH₃), sec-butyl (i.e. —CH(CH₃)CH₂CH₃), isobutyl (i.e. —CH₂CH(CH₃)₂) and tert-butyl (i.e. —C(CH₃)₃); and “propyl” includes n-propyl (i.e. —(CH₂)₂CH₃) and isopropyl (i.e. —CH(CH₃)₂).

“Alkenyl” refers to an alkyl group containing at least one carbon-carbon double bond and having from 2 to 20 carbon atoms (i.e., C220 alkenyl). 2 to 8 carbon atoms (i.e., C₂₋₈ alkenyl), 2 to 6 carbon atoms (i.e., C₂₋₆ alkenyl), or 2 to 4 carbon atoms (i.e., C₂₋₄ alkenyl). Examples of alkenyl groups include ethenyl, propenyl, butadienyl (including 1,2-butadienyl and 1,3-butadienyl).

“Alkoxy” refers to a group-OR where R is alkyl as defined herein.

“Aryl” refers to an aromatic carbocyclic group having a single ring (e.g. monocyclic) or multiple rings (e.g. bicyclic or tricyclic) including fused systems. As used herein, aryl has 6 to 20 ring carbon atoms (i.e., C₆₋₂₀ aryl), 6 to 12 carbon ring atoms (i.e., C₆₋₁₂ aryl), or 6 to 10 carbon ring atoms (i.e., C₆₋₁₈ aryl). Examples of aryl groups include phenyl. naphthyl. fluorenyl, and anthryl. Aryl, however, does not encompass or overlap in any way with heteroaryl defined below. If one or more aryl groups are fused with a heteroaryl. the resulting ring system is heteroaryl. If one or more aryl groups are fused with a heterocyclyl, the resulting ring system is heterocyclyl.

“Cycloalkyl” refers to a saturated or partially unsaturated cyclic alkyl group having a single ring or multiple rings including fused, bridged, and spiro ring systems. The term “cycloalkyl” includes cycloalkenyl groups (i.e. the cyclic group having at least one double bond). As used herein, cycloalkyl has from 3 to 20 ring carbon atoms (i.e., C₃₋₂₀ cycloalkyl), 3 to 12 ring carbon atoms (i.e., C₃₋₁₂ cycloalkyl), 3 to 10 ring carbon atoms (i.e., C₃₋₁₀ to cycloalkyl), 3 to 8 ring carbon atoms (i.e., C₃₋₈ cycloalkyl), or 3 to 6 ring carbon atoms (i.e., C₃₋₆ cycloalkyl). Examples of cycloalkyl groups include cyclopropyl, cyclobutyl, cyclopentyl, and cyclohexyl.

“Carboxylic acid” refers to an organic acid comprising the group —COOH “Alkali metal salt of carboxylic acid” refers to salts of carboxylic acids comprising Group I metal ions, i.e., lithium, sodium, potassium, rubidium, caesium, or francium salts.

The terms “optional” or “optionally” means that the subsequently described event or circumstance may or may not occur, and that the description includes instances where said event or circumstance occurs and instances in which it does not. Also, the term “optionally substituted” refers to any one or more hydrogen atoms on the designated atom or group may or may not be replaced by a moiety other than hydrogen.

Some of the compounds may exist as stereoisomers. Regardless of which stereoisomer is shown, the compounds are understood by one of ordinary skill in the art to include other stereoisomers and/or racemic mixtures. For example, if an (S) stereoisomer is shown, the (R) stereoisomer and the racemic mixture are also expressly included in the scope of embodiments presented herein.

Some of the compounds may exist as tautomers. Tautomers are in equilibrium with one another. For example, amide containing compounds may exist in equilibrium with imidic acid tautomers. Regardless of which tautomer is shown, and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

Any formula or structure given herein, is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl and ¹²⁵I. Various isotopically labeled compounds of the present disclosure, for example those into which radioactive isotopes such as ³H and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The disclosure also includes “deuterated analogs” of the compound of Formula I in which from 1 to n hydrogens attached to a carbon atom is/are replaced by deuterium, in which n is the number of hydrogens in the molecule. Such compounds exhibit increased resistance to metabolism and are thus useful for increasing the half-life of any compound of Formula I when administered to a mammal. particularly a human. See, for example, Foster, “Deuterium Isotope Effects in Studies of Drug Metabolism.” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An 18F labeled compound may be useful for PET or SPECT studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in the compound of Formula I.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

In many cases, the compounds of this disclosure are capable of forming acid and/or base salts by virtue of the presence of amino and/or carboxyl groups or groups similar thereto. A “salt” may be derived from an inorganic acid, an inorganic base, an organic acid, or an organic base. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, mandelic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, tetrahydrofuran carboxylic acid, and the like. Salts derived from inorganic bases include, by way of example only, sodium, potassium, lithium, ammonium, calcium and magnesium salts. Salts derived from organic bases include, but are not limited to, salts of primary, secondary and tertiary amines.

Provided are also pharmaceutically acceptable salts, hydrates, solvates, and tautomeric forms of Compound (I) described herein. “Pharmaceutically acceptable” or “physiologically acceptable” refer to compounds, salts, compositions, dosage forms and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound, and which are not biologically or otherwise undesirable. “Pharmaceutically acceptable salts” or “physiologically acceptable salts” include. for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid. fumaric acid. tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines, such as alkyl amines (i.e., NH₂(alkyl)), dialkyl amines (i.e., HN(alkyl)₂), trialkyl amines (i.e., N(alkyl)₃), substituted alkyl amines (i.e., NH₂(substituted alkyl)), di(substituted alkyl) amines (i.e., HN(substituted alkyl)₂), tri(substituted alkyl) amines (i.e., N(substituted alkyl)₃), alkenyl amines (i.e., NH₂(alkenyl)), dialkenyl amines (i.e., HN(alkenyl)₂), trialkenyl amines (i.e., N(alkenyl)₃), substituted alkenyl amines (i.e., NH₂(substituted alkenyl)), di(substituted alkenyl) amines (i.e., HN(substituted alkenyl)₂), tri(substituted alkenyl) amines (i.e., N(substituted alkenyl)₃, mono-, di-or tri-cycloalkyl amines (i.e., NH₂(cycloalkyl), HN(cycloalkyl)₂, N(cycloalkyl)₃), mono-, di-or tri-arylamines (i.e., NH₂(aryl), HN(aryl)₂, N(aryl)₃), or mixed amines, etc. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

A salt or pharmaceutically acceptable salt provided herein may be a “solvate” formed by the interaction of a solvent and a compound. Solvates of salts of the compounds described herein are also provided. Where the solvent is water, the solvate is a hydrate. A salt or pharmaceutically acceptable salt provided herein may be a hydrate. “Hydrates” of the compounds described herein are also provided.

The term “substantially crystalline” refers to forms of the compound of Formula (I) in which it is from 50% to 100% crystalline. Within this range, the compound of formula (I) may be at least 55% crystalline, or at least 60% crystalline, or at least 70% crystalline, or at least 80% crystalline, or at least 90% crystalline, or at least 95% crystalline, or at least 98% crystalline, or at least 99% crystalline, or at least 99.5% crystalline, or at least 99.9% crystalline, for example 100% crystalline.

The term “transaminase” refers to an amine transaminase (ATA) enzymatic reagent that is capable of transferring an amino group onto a suitable substrate. Where the substrate is pro-chiral, the transminase can selectively form a single stereoisomer. By way of example only, an amine transferase reagent can convert a ketone to either the (R) or (S) amine as shown below.

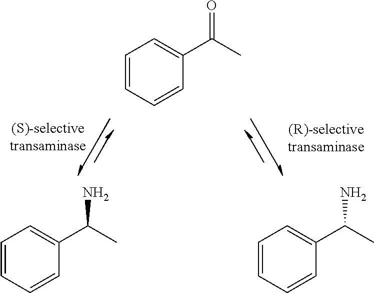

Examples of ATAs include and are not limited to commercially available ATAs such as (R—) selective transaminases: ATA-013, ATA-205, ATA-301, ATA-303, and ATA-412. Other ATAs included within the scope of this disclosure are commercially available, for instance, from the CODEX® ATA screening kit, the Johnson Matthey screening kit, Enzymeworks, Syncozymes, and the like, and are known to one of skill in the art.

Abbreviations

| Abbreviation | Meaning |
|---|---|
| ACN or MeCN | Acetonitrile |
| BINAP | (1,1′-Binaphthalene-2,2′-diyl)bis(diphenylphosphine) |
| | (2,2′-bis(diphenylphosphino)-1,1′-binaphthyl) |
| BuOH | n-Butanol |
| ° C. | Degree Celsius |
| DCM | Dichloromethane |
| DMF | Dimethylformamide |
| DIBAL-H | Diisobutylaluminum hydride |
| DIPEA | Diisopropylethylamine |
| aq. | Aqueous |
| g | Grams |
| hrs | Hours |
| M | Molar |
| Me | Methyl (CH₃) |
| MeOH | Methanol |
| mg | Milligram |
| MHz | Megahertz |
| ml/mL | Milliliter |
| mM | Millimolar |
| mmol | Millimole |
| MTBE | Methyl tert butyl ether |
| nL | Nanoliter |
| nm | Nanometer |
| μL/μl | Microliter |
| μM | Micromolar |
| Pd(dppf)Cl₂ | Bis(diphenylphosphino)ferrocene]dichloropalladium(II) |
| Pd₂(dba)₃ | Tris(dibenzylideneacetone)dipalladium(0) |
| PPh₃ | Triphenyl phosphine |
| P(o-Tol)₃, | Tri (o-tolyl) phosphine |
| PCy₃HBF₄ | Tricyclohexylphosphine tetrafluoroborate |
| dppf | 1,1′-Ferrocenediyl-bis(diphenylphosphine) |
| dppe | Ethylenebis(diphenylphosphine) |
| STAB | sodium triacetoxy borohydride |
| TBTU | 2-(1H-Benzotriazole-1-yl)-1,1,3,3-tetramethylaminium |
| | tetrafluoroborate |
| t-BuXphos | [2-Di-tert-butylphosphino-2′,4′,6′-triisopropylbiphenyl] |
| TFA | Trifluoroacetic acid |
| THF | Tetrahydrofuran |
| TLC | Thin layer chromatography |
| Xantphos | 4,5-Bis(diphenylphosphino)-9,9-dimethylxanthene |
| XPhos | 2-Dicyclohexylphosphino-2′,4′,6′-triisopropylbiphenyl |

### Methods

Provided herein is a process for preparing a compound of Formula (D), or a salt thereof,

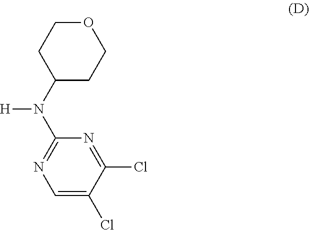 [SMILES: Clc1cnc(NC2CCOCC2)nc1Cl]

comprising
(i) contacting a compound of Formula (A), or a salt thereof,

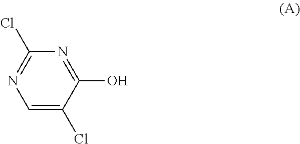

with a compound of Formula (B), or a salt thereof,

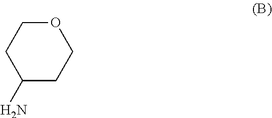

under conditions sufficient to provide a compound of Formula (C), or a salt thereof,

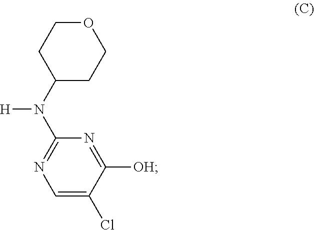 [SMILES: Oc1nc(NC2CCOCC2)ncc1Cl]

and
(ii) chlorinating the compound of Formula (C), or a salt thereof, to provide the compound of Formula (D) or a salt thereof.

In some embodiments of the process for preparing Compound (D), the compound of Formula (A) is a compound of Formula (A-1):

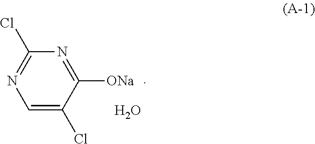

In some embodiments of the process for preparing Compound (D), step (i) further comprises a base and a solvent. In some of these embodiments, the base is an amine. In some embodiments, the amine is diisopropylethylamine. In some embodiments of the process for preparing Compound (D), the solvent is a protic solvent. In some embodiments, the protic solvent is n-butanol. Other protic solvent such a methanol, ethanol, isopropanol, propanol are contemplated within the scope of embodiments presented herein.

In some embodiments of the process for preparing Compound (D), step (ii) is conducted in the presence of phosphoryl chloride. Any other suitable chlorinating agent, e.g., sulfuryl chloride, thionyl chloride, phosgene and its derivatives (e.g., di and triphosgene, is contemplated within the scope of embodiments presented herein.

Provided herein is a process for preparing a compound of Formula (J) or a stereoisomer thereof, or a salt thereof,

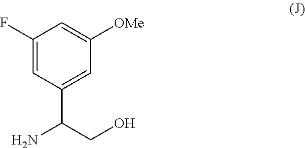 [SMILES: COc1cc(F)cc(C(N)CO)c1]

comprising
(i) contacting a compound of Formula (E)

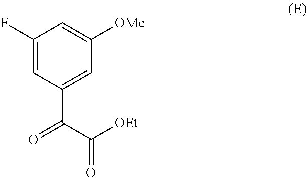 [SMILES: CCOC(=O)C(=O)c1cc(F)cc(OC)c1]

with a compound of Formula (F) or a stereoisomer thereof, or a salt thereof,

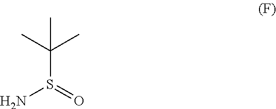

under conditions sufficient to provide a compound of Formula (G) or a stereoisomer thereof, or a salt thereof,

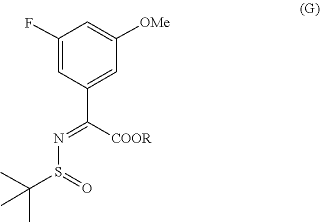

wherein R is C₁₋₅ alkyl; and
(ii) contacting the compound of Formula (G) or a stereoisomer thereof, or a salt thereof, with a reducing agent to provide the compound of Formula (H) or a stereoisomer thereof, or a salt thereof,

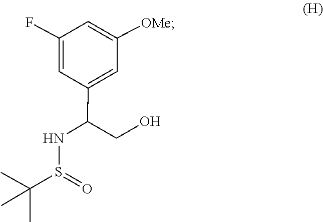 [SMILES: COc1cc(F)cc(C(CO)NS(=O)C(C)(C)C)c1]

and
iii) contacting the compound of Formula (H) or a stereoisomer thereof, or a salt thereof, with an acid to provide the compound of Formula (J) or a stereoisomer thereof, or a salt thereof.

In some embodiments of the process for preparing Compound (J),
the compound of Formula (F) has the structure of Formula (F-1):

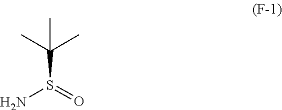

or a salt thereof, and
the compound of Formula (G) has the structure of Formula (G-1):

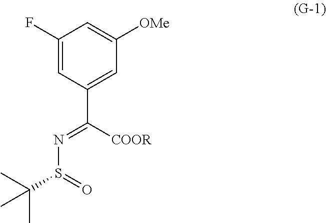

or a salt thereof;
wherein R is C₁₋₅ alkyl;
   the compound of Formula (H) has the structure of Formula (H-1):

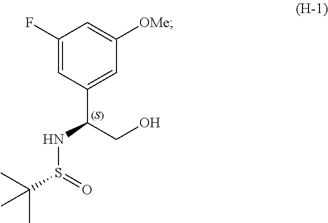

and
the compound of Formula (J) has the structure of Formula (J-1):

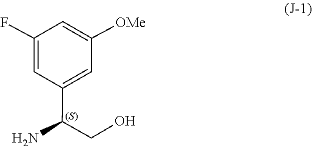

or a salt thereof.

Provided herein is a process for preparing a compound of Formula (J) or a stereoisomer thereof, or a salt thereof,

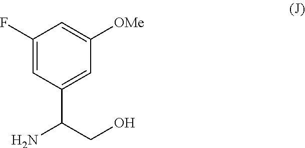 [SMILES: COc1cc(F)cc(C(N)CO)c1]

comprising
(i) contacting a compound of Formula (E)

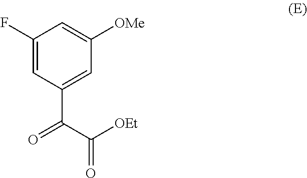 [SMILES: CCOC(=O)C(=O)c1cc(F)cc(OC)c1]

with a compound of Formula (F) or a stereoisomer thereof, or a salt thereof,

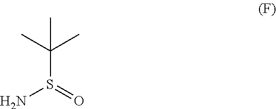

under conditions sufficient to provide a compound of Formula (G) or a stereoisomer thereof, or a salt thereof,

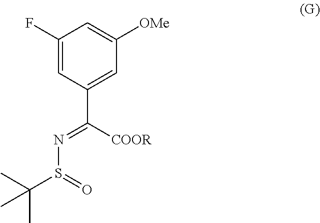

wherein R is C₁₋₅ alkyl;
(ii) contacting the compound of Formula (G) or a stereoisomer thereof, or a salt thereof, with a reducing agent to provide a compound of Formula (G-2), or a salt thereof,

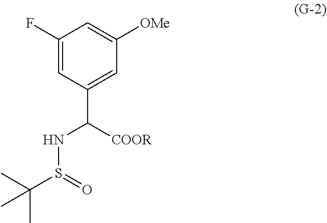

wherein R is C₁₋₅ alkyl;
(iii) contacting the compound of Formula (G-2) or a stereoisomer thereof, or a salt thereof, with a reducing agent to provide a compound of Formula (H) or a stereoisomer thereof, or a salt thereof,

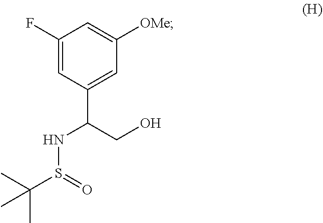 [SMILES: COc1cc(F)cc(C(CO)NS(=O)C(C)(C)C)c1]

and
iv) contacting the compound of Formula (H) or a stereoisomer thereof, or a salt thereof, with an acid to provide the compound of Formula (J) or a stereoisomer thereof, or a salt thereof.

In some embodiments of the process for preparing Compound (J),
the compound of Formula (F) has the structure of Formula (F-1):

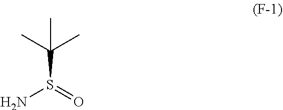

or a salt thereof, and
the compound of Formula (G) has the structure of Formula (G-1):

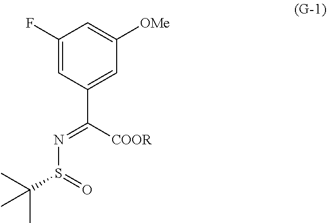

or a salt thereof;
wherein R is C₁₋₅ alkyl;
the compound of Formula (G-2) has the structure of Formula (G-3)

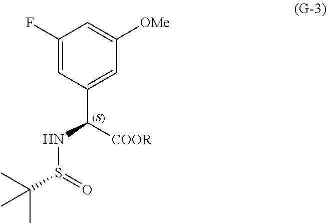

or a salt thereof,
wherein R is C₁₋₅ alkyl;
the compound of Formula (H) has the structure of Formula (H-1):

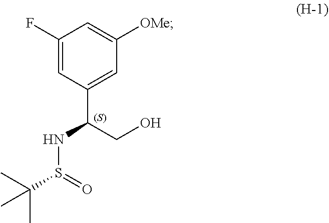

and
the compound of Formula (J) has the structure of Formula (J-1):

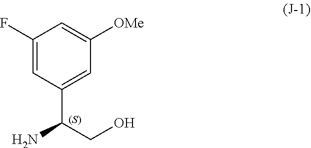

or a salt thereof.

In some embodiments of the process for preparing Compound (J), the compound of Formula (J) has the structure of Formula (J-2):

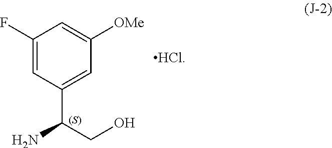

In some embodiments of the processes for preparation of Compound (J) described above, step (i) is conducted in the presence of a Lewis acid. In some embodiments, the Lewis acid is MgSO₄, CuSO₄, Cs₂CO₃, Yb(OTf)₃, ZnCl₂, tris-(2,2,2-trifluoro ethyl)borate, trialkyl borates, diazabicycloundecene (DBU), KO^{t}Bu, TiCl₄, BF₃.OEt₂, Sc(OTf)₃ or a titanium alkoxide of Formula (K):

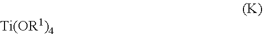

wherein R¹ is C₁₋₅ alkyl.

In some embodiments, the Lewis acid is Ti(OiPr)₄, or Ti(OEt)₄.

In some embodiments of the process for preparation of compound (J), a single reduction step reduces the imine bond and the ester group and the reducing agent in step (ii) is borane, NaBH₄/BF₃.OEt₂, sodium bis(2-methoxyethoxy)aluminum hydride (Red-Al®), diisobutylaluminium hydride (DIBAL), or NaBH₄/I₂. In some of such embodiments, the reducing agent in step (ii) is borane. In some of such embodiments, compound (G-1) is partially reduced and a mixture of compounds may be obtained wherein the ester group remains intact and/or is reduced to the alcohol.

In some embodiments of the process for preparation of compound (J), a first reduction step reduces the imine bond, and a second reduction step reduces the ester group, and the reducing agent for the first reduction in step (ii) is LiBH₄, NaBH₄, or ZnBH₄. In some of such embodiments, the reducing agent for the second reduction in step (iii) is borane, NaBH₄/BF₃.OEt₂, sodium bis(2-methoxyethoxy)aluminum hydride (Red-AIR), diisobutylaluminium hydride (DIBAL), or NaBH₄/I₂.

Provided herein is a process for preparing a compound of Formula (N), or a salt thereof,

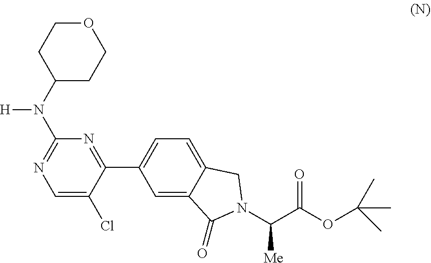 [SMILES: C[C@H](C(=O)OC(C)(C)C)N1Cc2ccc(-c3nc(NC4CCOCC4)ncc3Cl)cc2C1=O]

comprising
contacting a compound of Formula (D), or a salt thereof,

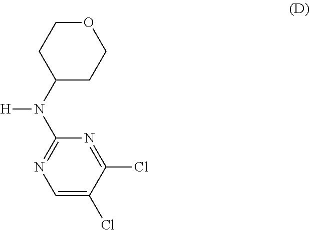 [SMILES: Clc1cnc(NC2CCOCC2)nc1Cl]

with a compound of Formula (M)

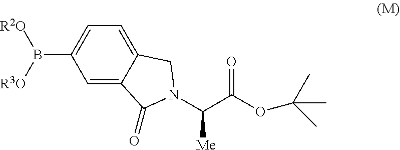

wherein R² and R³ are independently H, C₁₋₅ alkyl, or R² and R³ together with the atoms to which they are attached form a 5-or 6-membered ring optionally substituted with 1, 2, 3, or 4 C₁₋₃ alkyl; under conditions sufficient to provide a compound of Formula (N), or a salt thereof.

In some embodiments of the process for preparing compound (N), the process is conducted in the presence of an aqueous base and a palladium catalyst. In some of such embodiments, the aqueous base is aqueous K₂CO₃, aqueous Na₂CO₃, aqueous Cs₂CO₃, aqueous LiOH, or aqueous K₃PO₄. In some embodiments, the aqueous base is aqueous K3PO4. In some of these embodiments, the palladium catalyst is Pd(dppf)Cl₂, or Pd(OAc)₂ with a ligand selected from PPh₃, P(o-Tol)₃, PCy₃HBF₄, Dppf, Dppe, Xantphos, Xphos, BINAP (racemic, R, or S) and t-BuXphos. In some embodiments, the palladium catalyst is Pd(dppf)Cl₂.

In some embodiments of the process for preparing compound (N), the compound of Formula (D) is prepared by a process comprising
(i) contacting a compound of Formula (A), or a salt thereof,

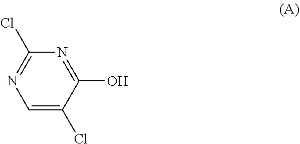

with a compound of Formula (B), or a salt thereof,

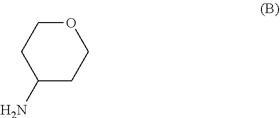

under conditions sufficient to provide a compound of Formula (C), or a salt thereof,

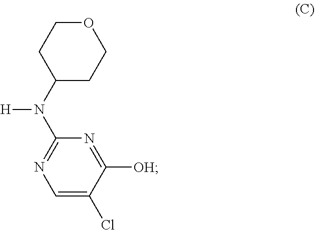 [SMILES: Oc1nc(NC2CCOCC2)ncc1Cl]

and
(ii) chlorinating the compound of Formula (C), or a salt thereof, to provide the compound of Formula (D), or a salt thereof.

In some embodiments of the process for preparing compound (N), the compound of Formula (A) is a compound of Formula (A-1):

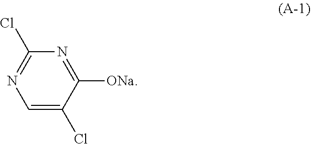

In some embodiments, the process for preparing compound (N) further comprises
(iv) removing the tert-butyl group in the compound of Formula (N), or a salt thereof, to provide a compound of Formula (O), or a salt thereof,

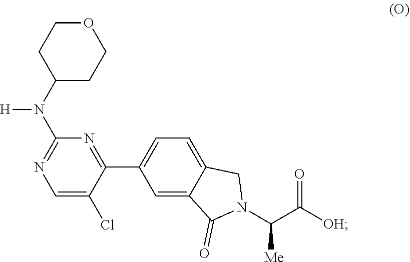 [SMILES: C[C@H](C(=O)O)N1Cc2ccc(-c3nc(NC4CCOCC4)ncc3Cl)cc2C1=O]

and
(v) coupling the compound of Formula (O) with a compound of Formula (J-1), or a salt thereof,

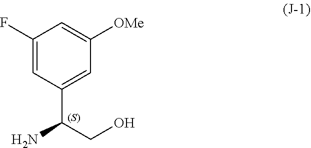

to provide a compound of Formula (I), or a pharmaceutically acceptable salt, solvate, or hydrate thereof,

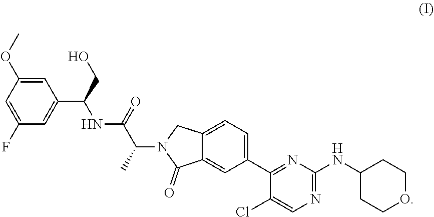 [SMILES: COc1cc(F)cc([C@@H](CO)NC(=O)[C@@H](C)N2Cc3ccc(-c4nc(NC5CCOCC5)ncc4Cl)cc3C2=O)c1]

In such embodiments of the process for preparing compound (N), step (v) provides a monohydrate of a compound of Formula (I).

In some embodiments, the compound of Formula (J-1), or a salt thereof, is prepared by a process comprising
(i) contacting a compound of Formula (E)

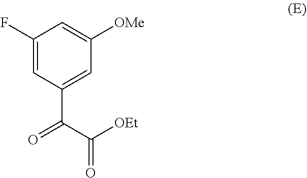 [SMILES: CCOC(=O)C(=O)c1cc(F)cc(OC)c1]

with a compound of Formula (F-1), or a salt thereof,

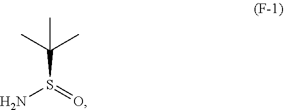

under conditions sufficient to provide a compound of Formula (G-1)

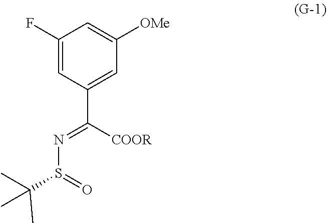

wherein R is C₁₋₅ alkyl;
(ii) contacting the compound of Formula (G-1) with a reducing agent to provide a compound of Formula (H-1), or a salt thereof,

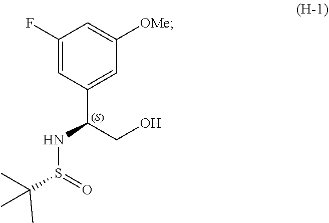

and
(iii) contacting the compound of Formula (H-1) with an acid to provide the compound of Formula (J-1), or a salt thereof.

In some embodiments, the compound of Formula (J-1), or a salt thereof, is prepared by a process comprising
(i) contacting a compound of Formula (E)

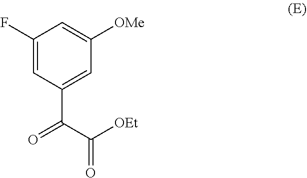 [SMILES: CCOC(=O)C(=O)c1cc(F)cc(OC)c1]

with a compound of Formula (F-1), or a salt thereof,

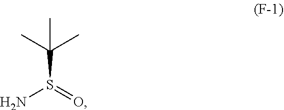

under conditions sufficient to provide a compound of Formula (G-1)

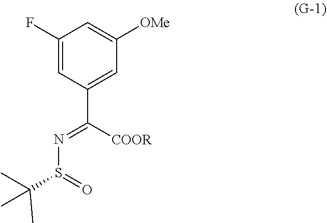

wherein R is C₁₋₅ alkyl;
(ii) contacting the compound of Formula (G-1) with a reducing agent to provide a compound of Formula (G-3), or a salt thereof

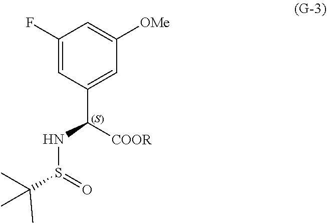

(iii) contacting the compound of Formula (G-3) with a reducing agent to provide a compound of Formula (H-1), or a salt thereof,

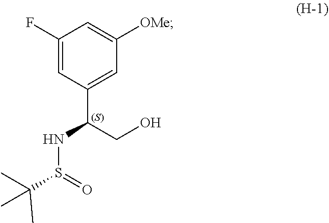

and
(iv) contacting the compound of Formula (H-1) with an acid to provide the compound of Formula (J-1), or a salt thereof.

In some embodiments, provided is a process for preparing a compound of Formula (AB)

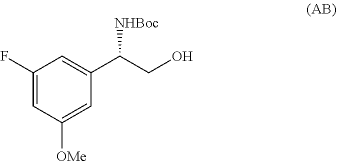 [SMILES: COc1cc(F)cc([C@@H](CO)NC(=O)OC(C)(C)C)c1]

comprising:
(i) contacting a compound of Formula (AA), or a salt thereof,

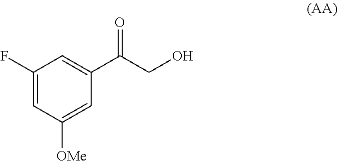 [SMILES: COc1cc(F)cc(C(=O)CO)c1]

with a transaminase to provide a compound of Formula (J-1), or a salt thereof,

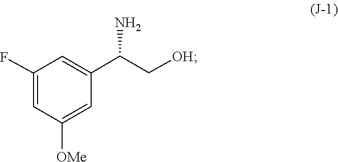 [SMILES: COc1cc(F)cc([C@H](N)CO)c1]

and
(ii) protecting the compound of Formula (J-1) to provide the compound of Formula (AB), wherein Boc is butyloxycarbonyl.

In some embodiments, the process further comprises deprotecting the compound of Formula (AB) to provide the compound of Formula (J-1), or a salt thereof:

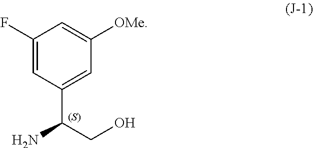

In some embodiments, the deprotecting is conducted in the presence of hydrochloric acid, trifluoro acetic acid, phosphoric acid, sulfuric acid, zinc bromide, catalytic iodine, acetyl chloride in methanol, or oxalyl chloride in methanol. In some embodiments, the deprotecting is conducted in the presence of hydrochloric acid and the compound of Formula (J-1) is a compound of Formula (J-2)

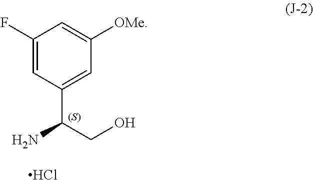

In some embodiments, the compound of Formula (AA) is prepared by contacting a compound of Formula (AC), or a salt thereof,

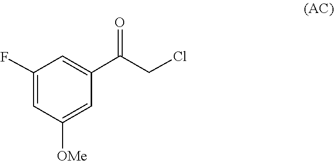 [SMILES: COc1cc(F)cc(C(=O)CCl)c1]

with alkali metal salts of carboxyclic acids, or carboxylic acids, or mixtures thereof, in the presence of a solvent and water. In some of such embodiments, the reaction is conducted in the presence of sodium formate and formic acid. Other alkali metal salts of carboxylic acids are known to one of skill in the art and are contemplated within the scope of the disclosure. Other carboxylic acids are known to one of skill in the art and are contemplated within the scope of the disclosure. In some embodiments, the solvent is an alcohol (e.g., methanol, ethanol, isopropanol), tetrahydrofuran, dimethylformamide, dimethylsulfoxide, acetonitrile, or a mixture thereof. In some embodiments the solvent is a mixture of an alcohol and one or more of tetrahydrofuran, dimethylformamide, dimethylsulfoxide, or acetonitrile.

In some embodiments, the compound of Formula (AC) is prepared by contacting a compound of Formula (AD)

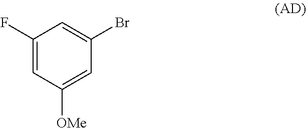

with a compound of Formula (AE), or a salt thereof,

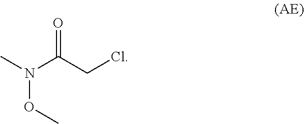

under conditions sufficient to provide the compound of Formula (AC).

Provided herein is a process for preparing a compound of Formula (J-2)

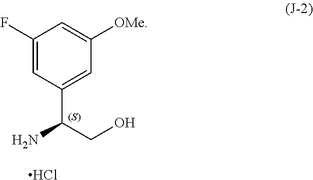

comprising:
(i) contacting a compound of Formula (AD)

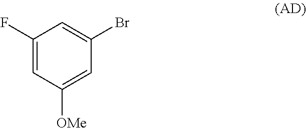

with magnesium metal and diethyl oxalate to provide a compound of Formula (E)

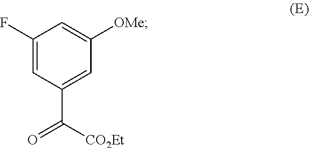 [SMILES: CCOC(=O)C(=O)c1cc(F)cc(OC)c1]

(ii) hydrolyzing the compound of Formula (E) to obtain a compound of Formula (AF), or a salt

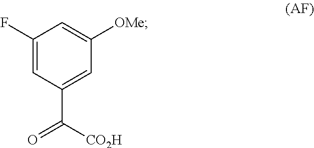 [SMILES: COc1cc(F)cc(C(=O)C(=O)O)c1]

thereof,
(iii) contacting the compound of Formula (AF), or a salt thereof, with a transaminase to provide a compound of Formula (AG), or a salt thereof,

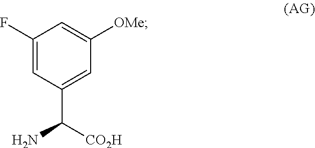 [SMILES: COc1cc(F)cc([C@H](N)C(=O)O)c1]

and
(iv) contacting the compound of Formula (AG) with a reducing agent and quenching the reaction with hydrochloric acid to provide the compound of Formula (J-2).

In some embodiments, the reducing agent is sodium borohydride and the reaction is conducted in the presence of a Lewis acid (e.g., boron trifluoride etherate BF₃.OEt₂). In some embodiments, the reducing agent is borane. In some embodiments the borane is generated in situ. Other suitable reducing agents are known to one of skill in the art and are contemplated within the scope of this disclosure.

In some embodiments, provided herein is a compound of Formula (AH):

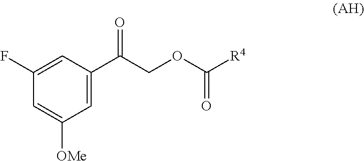

wherein R⁴ is H, C₂₋₆ alkyl or aryl. In some embodiments, compound AH is an intermediate formed in a reaction of compound (AC) with sodium formate. In some embodiments, R⁴ is H, C₁₋₆ alkyl or aryl.

Provided herein is a compound of Formula (AB), or a salt thereof

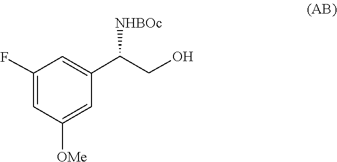

wherein Boc is butyloxycarbonyl.

Provided herein is a process for preparing a compound of Formula (I), or a pharmaceutically acceptable salt, solvate, or hydrate thereof,

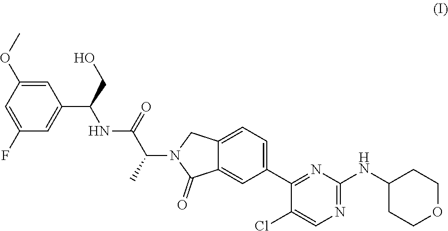 [SMILES: COc1cc(F)cc([C@@H](CO)NC(=O)[C@@H](C)N2Cc3ccc(-c4nc(NC5CCOCC5)ncc4Cl)cc3C2=O)c1]

comprising
(i) contacting a compound of Formula (A-1),

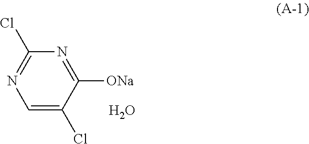

with a compound of Formula (B), or a salt thereof,

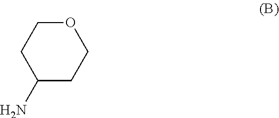

under conditions sufficient to provide a compound of Formula (C), or a salt thereof,

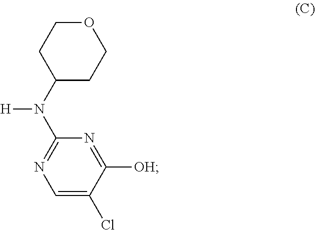 [SMILES: Oc1nc(NC2CCOCC2)ncc1Cl]

(ii) chlorinating the compound of Formula (C), or a salt thereof, to provide a compound of Formula (D), or a salt thereof,

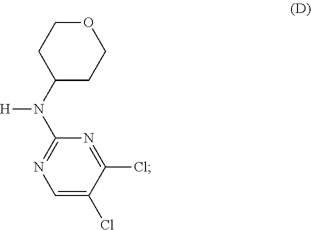 [SMILES: Clc1cnc(NC2CCOCC2)nc1Cl]

(iii) contacting the compound of Formula (D), or a salt thereof, with a compound of Formula (M)

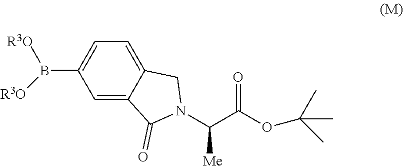

wherein R² and R³ are independently H, C₁₋₅ alkyl, or R² and R³ together with the atoms to which they are attached form a 5-or 6-membered ring optionally substituted with 1, 2, 3, or 4 C₁₋₃ alkyl; under conditions sufficient to provide a compound of Formula (N), or a salt thereof,

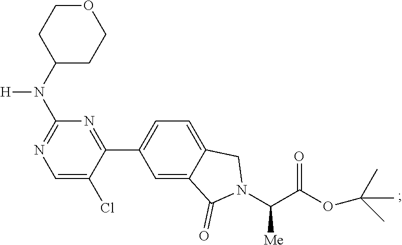 [SMILES: C[C@H](C(=O)OC(C)(C)C)N1Cc2ccc(-c3nc(NC4CCOCC4)ncc3Cl)cc2C1=O]

(iv) removing the tert-butyl group in the compound of Formula (N), or a salt thereof, to provide a compound of Formula (O), or a salt thereof,

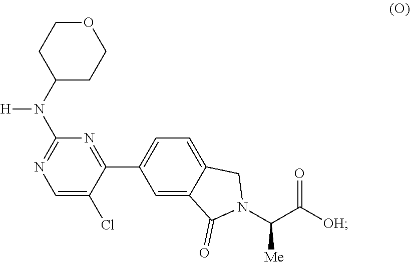 [SMILES: C[C@H](C(=O)O)N1Cc2ccc(-c3nc(NC4CCOCC4)ncc3Cl)cc2C1=O]

and
(v) coupling the compound of Formula (O) with a compound of Formula (J-2)

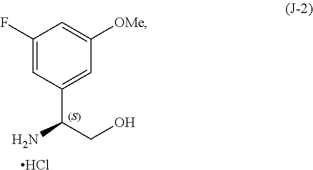

to provide the compound of Formula (I), or a pharmaceutically acceptable salt, solvate, or hydrate thereof;
wherein, in some embodiments, the compound of Formula (J-2) is prepared by a process comprising
(v-i) contacting a compound of Formula (E)

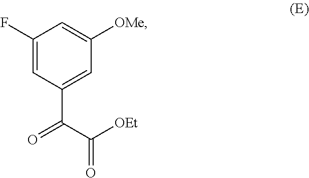 [SMILES: CCOC(=O)C(=O)c1cc(F)cc(OC)c1]

with a compound of Formula (F-1), or a salt thereof,

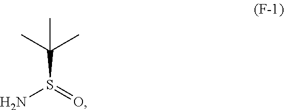

under conditions sufficient to provide a compound of Formula (G-1)

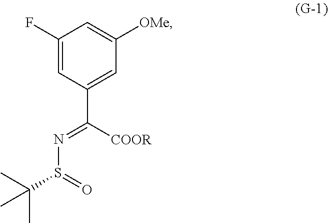

wherein R is C₁₋₅ alkyl;
(v-ii) contacting the compound of Formula (G-1) with a reducing agent to provide a compound of Formula (H-1), or a salt thereof,

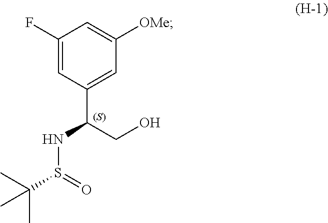

and
(v-iii) contacting the compound of Formula (H-1), or a salt thereof, with an acid to provide the compound of Formula (J-2).

In some embodiments of the process for preparing the compound of Formula (I) described above, the compound of Formula (J-2) is prepared as described in Example 19. In some embodiments of the process for preparing the compound of Formula (I) described above, the compound of Formula (J-2) is prepared as described in Example 20.

Provided herein is a process for preparing a compound of Formula (I), or a pharmaceutically acceptable salt, solvate, or hydrate thereof,

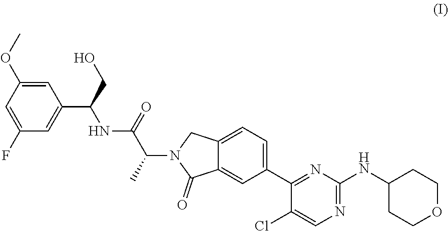 [SMILES: COc1cc(F)cc([C@@H](CO)NC(=O)[C@@H](C)N2Cc3ccc(-c4nc(NC5CCOCC5)ncc4Cl)cc3C2=O)c1]

comprising
(i) contacting a compound of Formula (A-1),

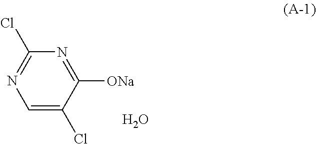

with a compound of Formula (B), or a salt thereof,

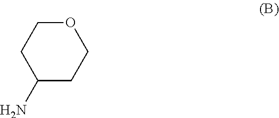

under conditions sufficient to provide a compound of Formula (C), or a salt thereof,

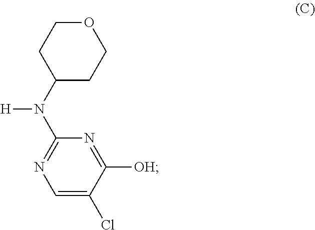 [SMILES: Oc1nc(NC2CCOCC2)ncc1Cl]

(ii) chlorinating the compound of Formula (C), or a salt thereof, to provide a compound of Formula (D), or a salt thereof,

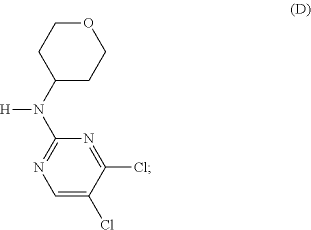 [SMILES: Clc1cnc(NC2CCOCC2)nc1Cl]

(iii) contacting the compound of Formula (D), or a salt thereof, with a compound of Formula (M)

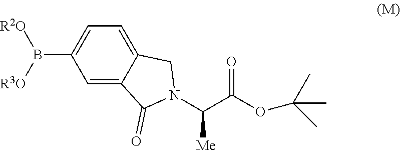

wherein R² and R³ are independently H, C₁₋₅ alkyl, or R² and R³ together with the atoms to which they are attached form a 5-or 6-membered ring optionally substituted with 1, 2, 3, or 4 C₁₋₃ alkyl; under conditions sufficient to provide a compound of Formula (N), or a salt thereof,

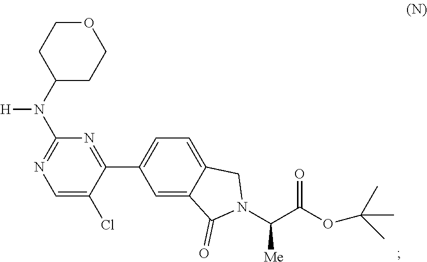 [SMILES: C[C@H](C(=O)OC(C)(C)C)N1Cc2ccc(-c3nc(NC4CCOCC4)ncc3Cl)cc2C1=O]

(iv) removing the tert-butyl group in the compound of Formula (N), or a salt thereof, to provide a compound of Formula (O), or a salt thereof,

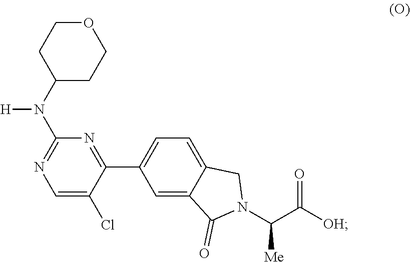 [SMILES: C[C@H](C(=O)O)N1Cc2ccc(-c3nc(NC4CCOCC4)ncc3Cl)cc2C1=O]

and
(v) coupling the compound of Formula (O) with a compound of Formula (J-2)

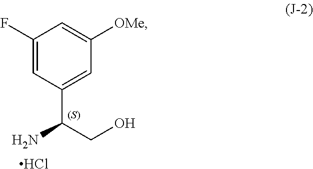

to provide the compound of Formula (I), or a pharmaceutically acceptable salt, solvate, or hydrate thereof;
wherein, in some embodiments, the compound of Formula (J-2) is prepared by a process comprising
(v-i) contacting a compound of Formula (E)

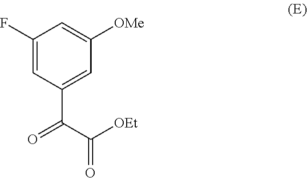 [SMILES: CCOC(=O)C(=O)c1cc(F)cc(OC)c1]

with a compound of Formula (F-1), or a salt thereof,

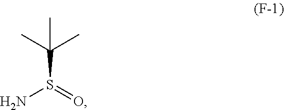

under conditions sufficient to provide a compound of Formula (G-1)

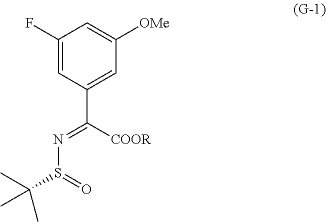

wherein R is C₁₋₅ alkyl;
(v-ii) contacting the compound of Formula (G-1) with a reducing agent to provide a compound of Formula (G-3), or a salt thereof,

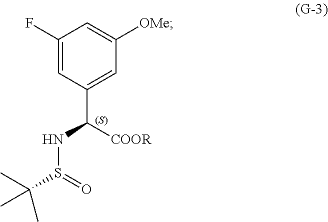

(v-iii) contacting the compound of Formula (G-3) with a reducing agent to provide a compound of Formula (H-1), or a salt thereof,

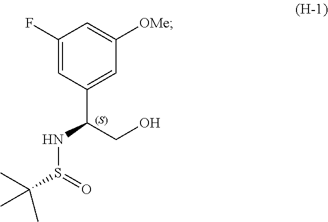

and
(v-iv) contacting the compound of Formula (H-1), or a salt thereof, with an acid to provide the compound of Formula (J-2).

In some embodiments of the process for preparing the compound of Formula (I) described above, the compound of Formula (J-2) is prepared as described in Example 19. In some embodiments of the process for preparing the compound of Formula (I) described above, the compound of Formula (J-2) is prepared as described in Example 20.

In some embodiments of the process for preparing the compound of Formula (I), step (v) provides a monohydrate of a compound of Formula (I).

For any process described herein, in some embodiments, the tert-butyl group is removed in the presence of trifluoroacetic acid (TFA).

Provided herein is a process for preparing a compound of Formula (L), or a salt thereof,

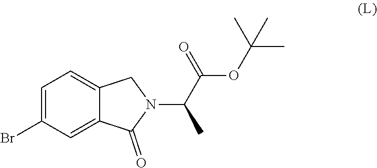 [SMILES: C[C@H](C(=O)OC(C)(C)C)N1Cc2ccc(Br)cc2C1=O]

comprising
contacting a compound of Formula (P)

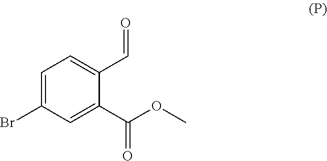

with a compound of Formula (Q), or a salt thereof,

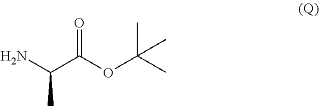

under conditions sufficient to provide the compound of Formula (L), or a salt thereof.

In some embodiments, the process for preparing compound (L) is conducted in the presence of sodium triacetoxy borohydride (STAB), a base, and a protic solvent. In some embodiments, the base is an amine.

Provided herein is a process for preparing a compound of Formula (N), or a salt thereof,

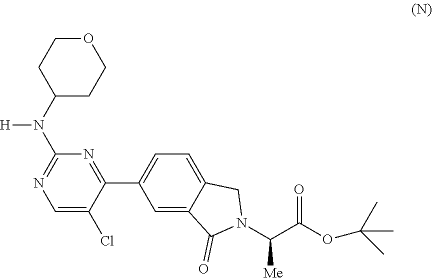 [SMILES: C[C@H](C(=O)OC(C)(C)C)N1Cc2ccc(-c3nc(NC4CCOCC4)ncc3Cl)cc2C1=O]

comprising
contacting a compound of Formula (L)

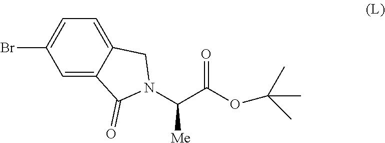 [SMILES: C[C@H](C(=O)OC(C)(C)C)N1Cc2ccc(Br)cc2C1=O]

with a compound of Formula (S)

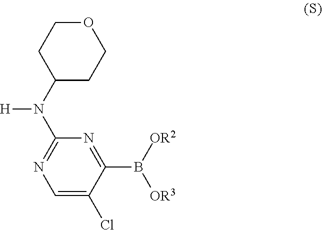

wherein R² and R³ are independently H, C₁₋₅ alkyl, or R² and R³ together with the atoms to which they are attached form a 5-or 6-membered ring optionally substituted with 1, 2, 3, or 4 C₁₋₃ alkyl; under conditions sufficient to provide the compound of Formula (N).

Provided herein is a process for preparing a compound of Formula (I), or a pharmaceutically acceptable salt, solvate, or hydrate thereof,

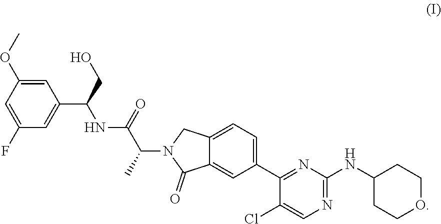 [SMILES: COc1cc(F)cc([C@@H](CO)NC(=O)[C@@H](C)N2Cc3ccc(-c4nc(NC5CCOCC5)ncc4Cl)cc3C2=O)c1]

comprising
contacting a compound of Formula (T), or a salt thereof,

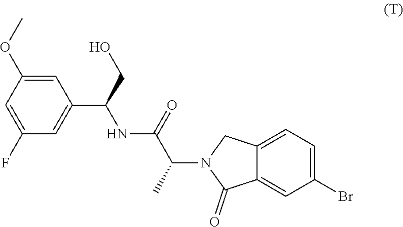 [SMILES: COc1cc(F)cc([C@@H](CO)NC(=O)[C@@H](C)N2Cc3ccc(Br)cc3C2=O)c1]

with a compound of Formula (S), or a salt thereof,

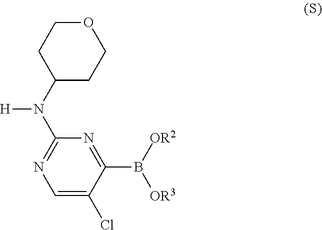

wherein R² and R³ are independently H, C₁₋₅ alkyl, or R² and R³ together with the atoms to which they are attached form a 5-or 6-membered ring optionally substituted with 1, 2, 3, or 4 C₁₋₃ alkyl; under conditions sufficient to provide the compound of Formula (I), or a pharmaceutically acceptable salt, solvate, or hydrate thereof.

Provided herein is a process for preparing a compound of Formula (I), or a pharmaceutically acceptable salt, solvate, or hydrate thereof,

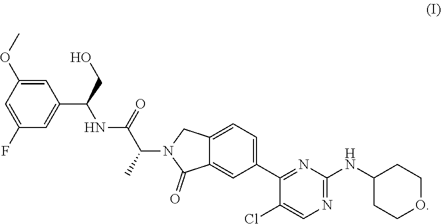 [SMILES: COc1cc(F)cc([C@@H](CO)NC(=O)[C@@H](C)N2Cc3ccc(-c4nc(NC5CCOCC5)ncc4Cl)cc3C2=O)c1]

comprising
contacting a compound of Formula (V), or a salt thereof,

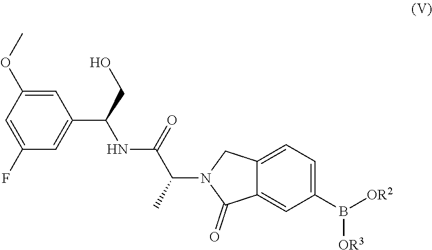

wherein R² and R³ are independently H, C₁₋₅ alkyl, or R² and R³ together with the atoms to which they are attached form a 5-or 6-membered ring optionally substituted with 1, 2, 3, or 4 C₁₋₃ alkyl;
with a compound of Formula (D), or a salt thereof,

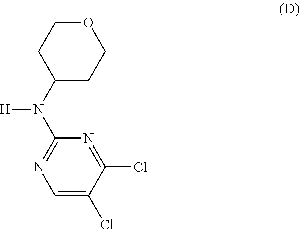 [SMILES: Clc1cnc(NC2CCOCC2)nc1Cl]

under conditions sufficient to provide the compound of Formula (I), or a pharmaceutically acceptable salt, solvate, or hydrate thereof.

In some embodiments, the compound of Formula (I) is a monohydrate.

Provided herein is Compound (I) prepared according to any process described herein.

Provided herein is a Compound (I), or hydrate thereof, prepared according to any process described herein. Provided herein is a Compound (I), or monohydrate thereof, prepared according to any process described herein.

In some embodiments, a monohydrate of a compound of Formula (1) is a crystalline form (Form B). Form B is described in WO 2018/193410. In some embodiments, provided herein are improved methods for preparing Form B of the compound of Formula (I). In some embodiments, Form B of Compound (I) can be characterized by an X-ray diffraction pattern exhibiting peaks of greatest intensity at the diffraction angles set out in Table A, i.e. 14.0°, 20.6°, 24.0°, and 24.2° (±0.2°).

TABLE A
Diffraction Angle (°)

14.0
20.6
24.0
24.2

The data collection and structure refinement was conducted as follows.

| | |
|---|---|
| Diffractomer | SuperNova, Dual, Cu at zero, Atlas |
| Radiation Source | SuperNoca (Cu) X-ray Source, CuKα |
| Data collection method | scans |
| Theta range for data collection | 3.460 to 66.589° |
| Index ranges | −14 ≤ h ≤ 15, −10 ≤ |
| | h ≤ 9, −14 ≤ h ≤ 15, |
| Reflections collected | 24752 |
| Independent reflections | 4834 [R(int) = 0.0371] |
| Coverage of independent | 97.4% |
| reflections | |
| Absorption correction | Semi-empirical from equivalents |
| Max. and min. transmission | 1.00000 and 0.80872 |
| Structure solution technique | Direct Methods |
| Structure solution/refinement | SHELXTL (Sheldrick, 2013) |
| program | |
| Refinement technique | Full-matrix lease-squares on F2 |

Some embodiments provide for Form B of Compound (I) having an X-ray powder diffraction pattern characterized by the presence of major peaks at the diffraction angles (2θ) 14.0° and/or 20.6° and/or 24.0° and/or 24.2° (±0.2°).

In some embodiments, the X-ray diffraction pattern of Form B of Compound (I) is characterized by the presence of at least one peak at a diffraction angle selected from 14.0°, 20.6°, 24.0°, and 24.2° (±0.2°). In some embodiments, provided herein is a substantially crystalline form (Form B) of Compound (I) having an X-ray powder diffraction pattern characterized by the presence of a major peak at the diffraction angle 14.0° (±0.2°). In some embodiments, provided herein is a substantially crystalline form (Form B) of Compound (I) having an X-ray powder diffraction pattern characterized by the presence of a major peak at the diffraction angle 20.6° (±0.2°). In some embodiments, provided herein is a substantially crystalline form (Form B) of Compound (I) having an X-ray powder diffraction pattern characterized by the presence of a major peak at the diffraction angle 24.0° (±0.2°). In some embodiments, provided herein is substantially crystalline form (Form B) of Compound (I) having an X-ray powder diffraction pattern characterized by the presence of a major peak at the diffraction angle 24.2° (±0.2°).

In some embodiments, a substantially crystalline form (Form B) of Compound (I) has an X-ray powder diffraction pattern characterized by the presence of major peaks at two or more, e.g. three or four diffraction angle, selected from 14.0°, 20.6°, 24.0°, and 24.2° (±0.2°).

The X-ray powder diffraction pattern of Form B of compound (I) may also have peaks present at the diffraction angles selected from 8.8, 13.0, 13.8, 14.4, 17.3, 19.3, 21.3, and 28.7 (±0.2°). Some embodiments provide for a substantially crystalline form (Form B) of Compound (I) having an X-ray powder diffraction pattern characterized by the presence of major peaks at the diffraction angles 14.0° and/or, 20.6° and/or 24.0° and/or 24.2° (±0.2° ) as defined above and optionally one or more further peaks at diffraction angles selected from 8.8°, 13.0°, 13.8°, 14.4°, 17.3°, 19.3°, 21.3°, and/or 28.7 ° (±0.2°). In some embodiments, the substantially crystalline form (Form B) of Compound (I) has an X-ray powder diffraction pattern characterized by the presence of major peaks at the diffraction angles 14.0° and/or 20.6° and/or 24.0° and/or 24.2° (±0.2°); and optionally one or more further peaks at the diffraction angles 13.8° and/or 9.3° and/or 21.3° (±0.2°).

In some embodiments, the substantially crystalline form (Form B) of compound (I) has an X-ray powder diffraction pattern characterized by the presence of major peaks at the diffraction angles 14.0°, 20.6°, 24.0°, 24.2°, 13.8°, 19.3°, and 21.3° (±0.2°).

In some embodiments, the substantially crystalline form (Form B) of compound (I) has an X-ray powder diffraction pattern characterized by the presence of major peaks at the diffraction angles 14.0°, 20.6°, 24.0°, 24.2°, 8.8°, 13.0°, 13.8°, 14.4°, 17.3°, 19.3°, 21.3°, and 28.7° (±0.2°).

In some embodiments, a substantially crystalline form (Form B) of Compound (I) exhibits an endothermic event having an onset temperature between 100° C. to 110° C. when subjected to differential scanning calorimetry (DSC). In some embodiments, a substantially crystalline form (Form B) of compound (I) exhibits an endothermic event having an onset temperature between 101° C. to 108° C. when subjected to DSC. Some embodiments provide for a substantially crystalline form (Form B) of compound (I) which exhibits an endothermic event having a peak between 110° C. and 125° C. Some embodiments provide for a substantially crystalline form (Form B) of compound (I) which exhibits an endothermic event having a peak between 111° C. and 113° C.

In some embodiments, the substantially crystalline Form B of compound (I) has been analyzed by thermogravimetric analysis (TGA) and exhibits a weight loss transition with an onset temperature of 85° C. to 95° C., for example 90.86° C. which is complete at 110° C. to 130° C., for example 120° C.

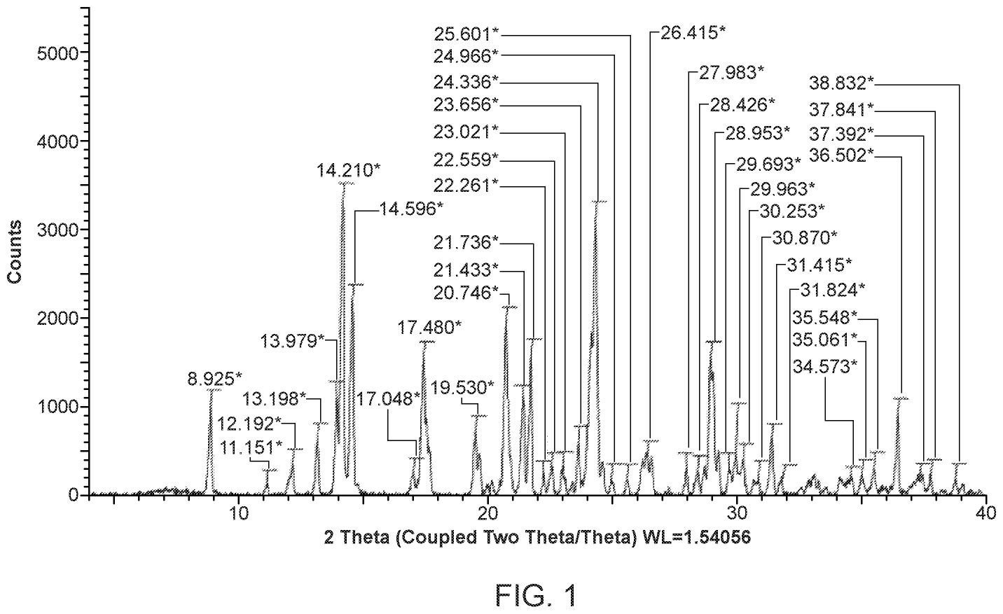

Provided herein is a substantially crystalline form (Form B) of Compound (I) prepared according to the methods described herein and having an X-ray powder diffraction pattern substantially as shown in FIG. 1. The data collection was conducted as follows.

| | |
|---|---|
| Diffractometer | Bruker D8 Focus |
| Vertical goiniometer | Theta/2 theta |
| Sample stage | Rotation |
| Filter | Nickel K_{β} filter |
| X-ray generator | Cu, Kα, λ = 1.54056 Å |
| Tube voltage | 40 kV |
| Tube current | 40 mA |
| Scan type | Locked coupled |
| Scan mode | Continuous |
| Scan range | 4°-40° 2 theta using Cu Kα radiation |
| Scan step | 0.02° |
| Step time | 0.3 s |
| Sample rotating rate | 15 rotations/minute |
| Detector | LYNXEYE |

In some embodiments, Form B of Compound (I) can be characterized by an X-ray diffraction pattern exhibiting peaks of greatest intensity at certain diffraction angles, i.e. 14.2°, 14.6°, 20.7°, and 24.3° (±0.2°). In some embodiments, Form B of Compound (I) can be characterized by an X-ray diffraction pattern exhibiting peaks of greatest intensity at certain diffraction angles, i.e. 8.9°, 14.0°, 14.2°, 14.6°, 20.7°, 24.3°, and 29.0° (±0.2°). In some embodiments, Form B of Compound (I) can be characterized by an X-ray diffraction pattern exhibiting peaks of greatest intensity at certain diffraction angles, i.e. 8.9°, 13.2°, 14.0°, 14.2°, 14.6°, 17.5°, 19.5°, 20.7°, 21.4°, 21.7°, 23.7°, 24.3°, and 29.0° (±0.2°). In some embodiments, Form B of Compound (I) can be characterized by an X-ray diffraction pattern exhibiting peaks of greatest intensity at the diffraction angles set forth in Table B.

TABLE B
Diffraction Angle

14.0°
14.2°
14.6°
20.7°
24.3°
29.0°

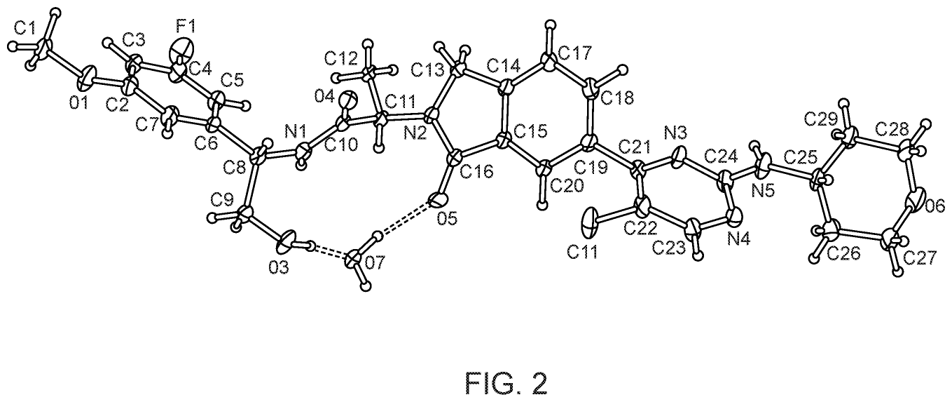

In some embodiments, provided herein is a substantially crystalline form (Form B) of Compound (I), wherein the Form B is a monohydrate of Compound (I). FIG. 2 shows a single crystal X-ray structure of Form B as an ORTEP plot.

Provided herein is a composition comprising a compound of Formula (I)

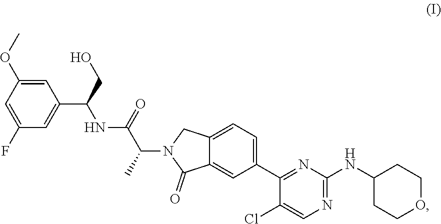 [SMILES: COc1cc(F)cc([C@@H](CO)NC(=O)[C@@H](C)N2Cc3ccc(-c4nc(NC5CCOCC5)ncc4Cl)cc3C2=O)c1]

wherein the composition comprises no more than 0.5% area/area of compounds of Formula (Z-1) and/or Formula (Z-2)

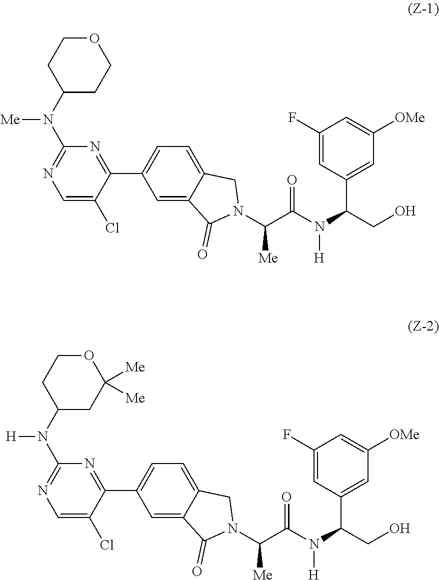 [SMILES: COc1cc(F)cc([C@@H](CO)NC(=O)[C@@H](C)N2Cc3ccc(-c4nc(N(C)C5CCOCC5)ncc4Cl)cc3C2=O)c1 ; COc1cc(F)cc([C@@H](CO)NC(=O)[C@@H](C)N2Cc3ccc(-c4nc(NC5CCOC(C)(C)C5)ncc4Cl)cc3C2=O)c1]

In one embodiment, Compound (I) prepared according to any process described herein comprises no more than 0.1% area/area of compounds (Z-1) and (Z-2. In one embodiment, Compound (I) prepared according to any process described herein comprises no more than 0.3% area/area of compound (Z-1). In one embodiment, Compound (I) prepared according to any process described herein comprises no more than 0.1% area/area of compound (Z-1). In one embodiment, Compound (I) prepared according to any process described herein comprises no more than 0.3% area/area of compound (Z-2). In one embodiment, Compound (I) prepared according to any process described herein comprises no more than 0.1% area/area of compound (Z-2). As used herein, “area/area” refers to the peak areas on an HPLC or a chiral HPLC.

(S)-2-amino-2-(3-fluoro-5-methoxyphenyl)ethan-1-ol hydrochloride (Compound (J-2), CAS Number : 2095692-22-9) is commercially available from Sigma Aldrich in small quantities in about 95% purity, although the chiral purity is unknown. Large quantities of this material in high chiral purity were desirable. Scheme 1 outlines an improved procedure for the preparation of compound (J-2).

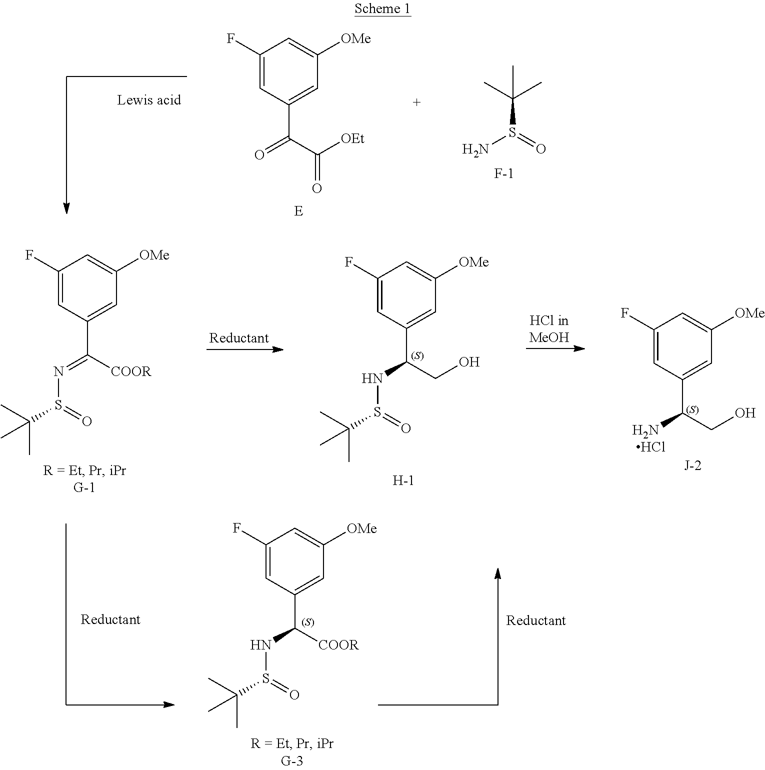

A Lewis acid mediated reaction between compound (E) and compound (F-1) provides the imine compound (G-1) which can be reduced to compound (H-1) in a single reduction step (e.g., by use of reductants such as borane, NaBH₄/BF₃.OEt₂, Sodium bis(2-methoxyethoxy)aluminum hydride (Red-Al®), diisobutylaluminium hydride (DIBAL), NaBH₄/I₂ or any other suitable reducing agent. Alternatively, the imine compound (G-1) can be reduced to compound (G-3) using a first reductant such as LiBH₄, NaBH₄, ZnBH₄ or any other suitable reducing agent, and then further reduced to compound (H-1) using an additional reductant such as borane, NaBH₄/BF₃.OEt₂, Sodium bis(2-methoxyethoxy)aluminum hydride (Red-Al®), diisobutylaluminium hydride (DIBAL), NaBH4/12 or any other suitable reducing agent. The Lewis acid may be MgSO₄, CuSO₄, Cs₂CO₃, Yb(OTf)₃, ZnCl₂, tris-(2,2,2-trifluoro ethyl)borate, trialkyl borates, diazabicycloundecene (DBU), KO^{t}Bu, TiCl₄, BF₃.OEt₂, Sc(OTf)₃ or a titanium alkoxide of Formula (K):

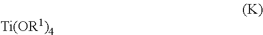

wherein R¹ is C₁₋₅ alkyl, or any other suitable Lewis acid.

As described in Example 10, the initial procedure for the preparation of Compound (J-2) provided a chiral HPLC purity of about 91.5% area for Compound (J-2).

In order to improve the chiral purity of Compound (J-2), Compound (J-2) was free-based to Compound (J-1) as described in Example 11.

The free base Compound (J-1) was converted to a mandelic acid salt, i.e., Compound (J-3), as described in Example 12. Compound (J-3) was then free based to provide Compound (J-1) as described in Example 14. The chiral purity of the free base, i.e., Compound (J-1) was now improved to 99.9% area. Compound (J-1) was then converted to the HCl salt, i.e., Compound (J-2) as described in Example 15. The chiral HPLC purity of Compound (J-2) was now improved to about 99.9% area as described in Example 15.

Similarly, the free base Compound (J-1) was converted to a furoate acid salt, i.e., Compound (J-4), as described in Example 13. Compound (J-4) was then converted to the HCl salt, i.e., Compound (J-2) as described in Example 13. The chiral HPLC purity of Compound (J-2) was now improved to about 100% area as described in Example 13.

Accordingly, the methods described herein provide an improved intermediate, i.e., Compound (J-2), having superior chiral purity.

Scheme 2 below shows an embodiment for the synthesis of Compound (I) using the methods described above and in the Examples section.

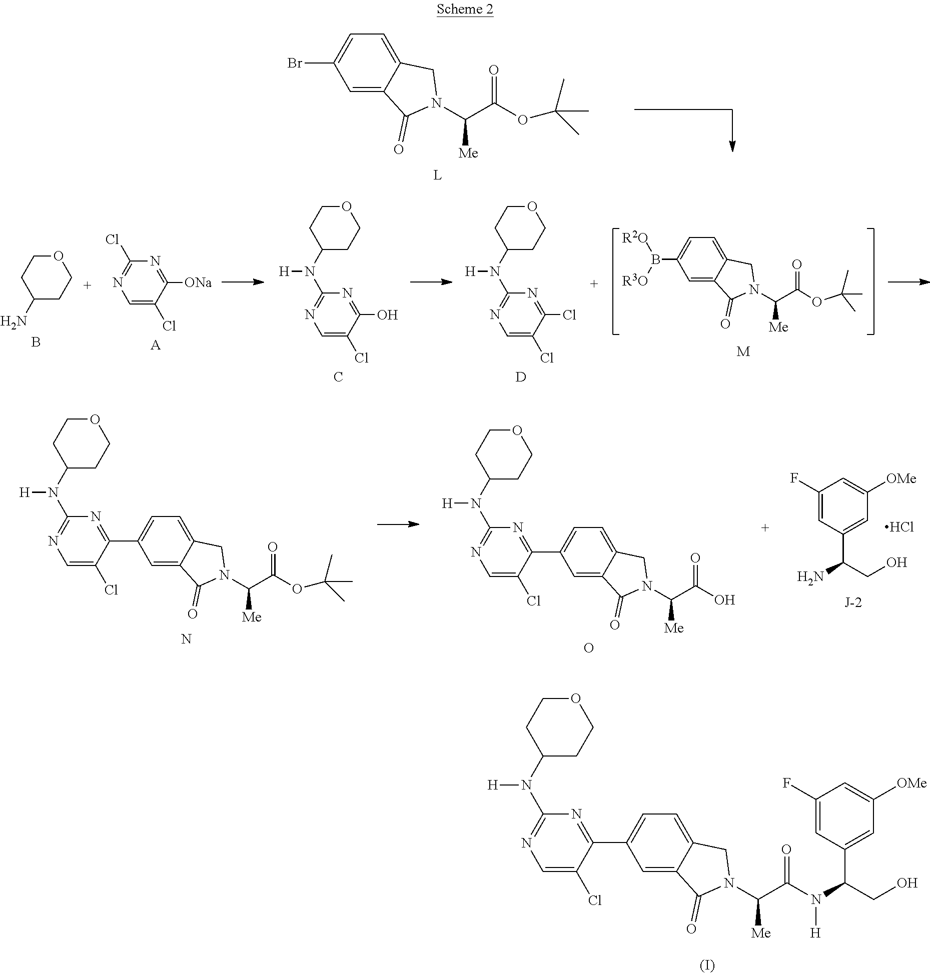

R² and R³ in Scheme 2 are as defined herein in some or any embodiments. Compound (L) is converted to boronate (M) in the presence of a borylating agent and a catalyst such as Pd(dppf)Cl₂, Pd₂(dba)₃, Pd(PPh₃)₄ or any other suitable catalyst for a metal-mediated coupling reaction. Suitable solvents for the reaction include and are not limited to acetonitrile, DMF, or other aprotic solvents. A base may be used, for instance, KOAc, NaOAc or any other suitable base. The reaction temperature may range from about 70° C, to 120° C., from about 100° C. to 120° C., or from about 80° C. to 90° C. After the formation of the boronate (M), Compound (D) is added to the reaction mixture at a lower temperature (e.g., by cooling the reaction mixture to about 70° C. to 85° C., or about 70° C. to 75° C.) in the presence of an aqueous base. Any suitable aqueous base may be used including and not limited to K₂CO₃, aqueous Na₂CO₃, aqueous Cs₂CO₃, aqueous LiOH, and/or aqueous K₃PO₄. NaOH and/or NaHCO₃ may also be used though it has been found that NaOH may cause racemization. The palladium catalyst from the first step remains in the reaction mixture and also catalyzes the reaction of compound (D) with compound (M) in a single pot process. It will be understood that the reactions may be also be conducted in separate steps/pots/reactors. Compound (N) formed according to Scheme 2 is then converted to Compound (I). Example 3 describes one embodiment for the preparation of compound (N) as shown in Scheme 2. The use of compound (D), and the use of compound (J-2) prepared according to the methods described herein, allows for an overall improved yield of Compound (I).

Scheme 3 below shows an embodiment for the synthesis of Compound (I) using the methods described above and in the Examples section.

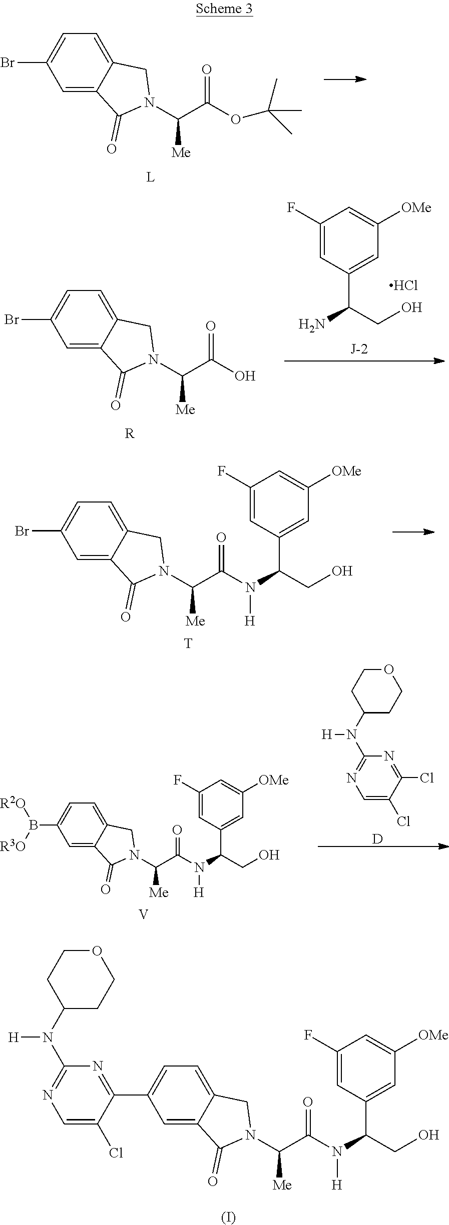

R² and R³ in Scheme 3 are as defined herein in some or any embodiments. Compound (T) is converted to boronate (V) in the presence of a borylating agent and a catalyst such as Pd(dppf)Cl₂, Pd₂(dba)₃, Pd(PPh₃)₄ or any other suitable catalyst for a metal-mediated coupling reaction. Suitable solvents for the reaction include and are not limited to acetonitrile, DMF, or other aprotic solvents. A base may be used, for instance, KOAc, NaOAc or any other suitable base. The reaction temperature may range from about 70° C. to 120° C., from about 100° C. to 120° C., or from about 80° C. to 90° C. After the formation of the boronate (V), Compound (D) is added to the same reaction mixture at a lower temperature (e.g., by cooling the reaction mixture to about 70° C. to 85° C., or about 70° C. to 75° C.) in the presence of an aqueous base. Any suitable aqueous base may be used including and not limited to K₂CO₃, aqueous Na₂CO₃, aqueous Cs₂CO₃, aqueous LiOH, and/or aqueous K₃PO₄. NaOH and/or NaHCO3 may also be used though it has been found that NaOH may cause racemization. Optionally, a catalyst may be added such as Pd(dppf)Cl₂, Pd₂(dba)₃, Pd(PPh₃)₄ or any other suitable catalyst for a metal-mediated coupling reaction with compound (D). Optionally the two reactions may be conducted as a one pot procedure wherein the catalyst from the first step also catalyzes the second reaction. Example 18 describes one embodiment for the preparation of compound (I) from compound (T). The use of compound (D), and the use of compound (J-2) prepared according to the methods described herein, allows for an overall improved yield of Compound (I).

Scheme 4 below shows an embodiment for the synthesis of Compound (I) using the methods described above and in the Examples section.

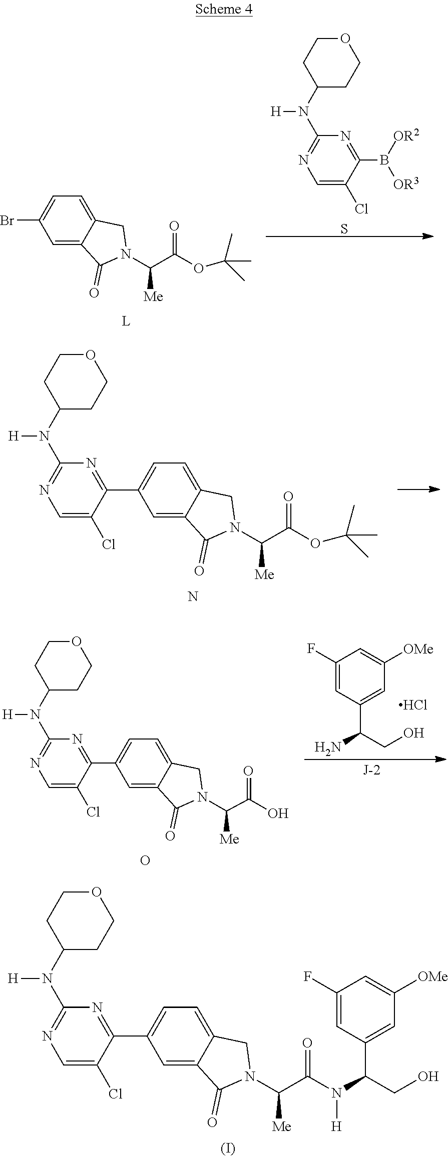

R² and R³ in Scheme 4 are as defined herein in some or any embodiments. Instead of compound (D), compound (S) may be used for coupling with the bromo compound (L) using standard coupling procedures as described herein or known to one of skill in the art. The use of compound (S), and the use of compound (J-2) prepared according to the methods described herein, allows for an overall improved yield of Compound (I). Compound (S) can be prepared starting from compound (D) using suitable borylating conditions known to one of skill in the art.

Scheme 5 below shows an embodiment for the synthesis of Compound (I) using the methods described above and in the Examples section.

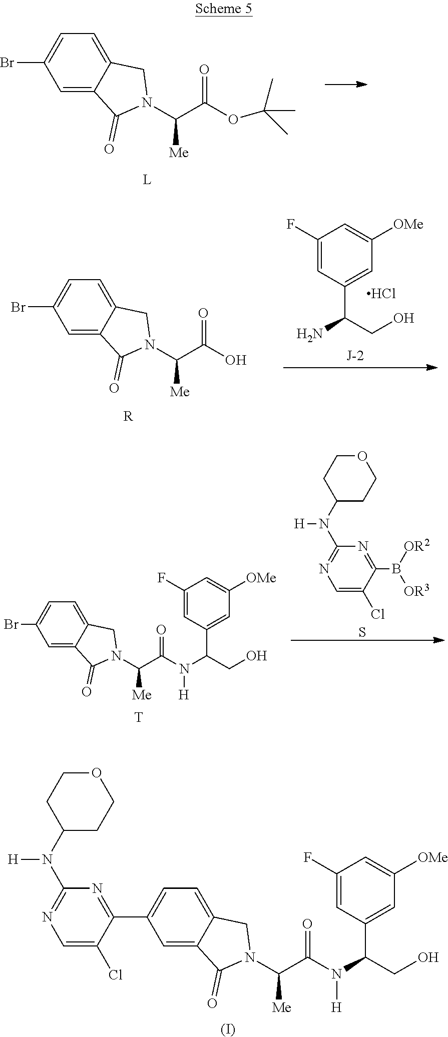

R² and R³ in Scheme 5 are as defined herein in some or any embodiments. Instead of compound (D), compound (S) may be used for coupling with the bromo compound (T) using standard coupling procedures as described herein or known to one of skill in the art. The use of compound (S), and the use of compound (J-2) prepared according to the methods described herein, allows for an overall improved yield of Compound (I).

Any combination of steps described above may be used in the preparation of compounds described herein, including any procedures described in the Examples section.

The compounds of this disclosure can be prepared from readily available starting materials using, for example, the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts (1999) Protecting Groups in Organic Synthesis, 3rd Edition, Wiley, New York, and references cited therein.

Furthermore, the compounds of this disclosure may contain one or more chiral centers. Accordingly, if desired, such compounds can be prepared or isolated as pure stereoisomers, i.e., as individual enantiomers or diastereomers or as stereoisomer-enriched mixtures. All such stereoisomers (and enriched mixtures) are included within the scope of this disclosure, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents, and the like.

The starting materials for the following reactions are generally known compounds or can be prepared by known procedures or obvious modifications thereof. For example, many of the starting materials are available from commercial suppliers such as Aldrich Chemical Co. (Milwaukee, Wisconsin, USA), Bachem (Torrance, California, USA), Emka-Chemie or Sigma (St. Louis, Missouri, USA). Others may be prepared by procedures or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989) organic Reactions, Volumes 1-40 (John Wiley, and Sons, 1991), March's Advanced Organic Chemistry, (John Wiley, and Sons, 5^{th} Edition, 2001), and Larock's Comprehensive Organic Transformations (VCH Publishers Inc., 1989).

### EXAMPLES

The compounds and intermediates described herein may be prepared using the methods disclosed herein and routine modifications thereof, which will be apparent given the disclosure herein and methods well known in the art. Conventional and well-known synthetic methods may be used in addition to the teachings herein. The synthesis of typical compounds described herein may be accomplished as described in the following examples. If available, reagents may be purchased commercially, e.g., from Sigma Aldrich or other chemical suppliers.

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Preparation of 5-chloro-2-[(tetrahydro-2H-pyran-4-yl)amino] pyrimidin-4-ol. compound of Formula (C)

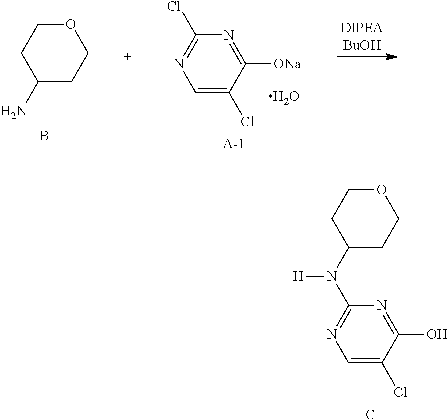

A mixture of compound of Formula (A-1) (85 Kg, 82.5% assay, as free base, 99.9% area purity, 1 eq), compound of Formula (B) (85.2 Kg, 2 eq) and diisopropylethylamine (219.8 Kg, 4 eq) in n-butanol (576.6 Kg) was heated to 110 to 115° C, for six days. After the consumption of compound of Formula A-1 to 0.80% area by HPLC, the reaction mixture was cooled to 20-30° C. The reaction mixture was extracted with 3×386 Kg of 10% K₃PO₃ aqueous solution. The combined aqueous layer was washed with ethyl acetate (282 Kg). After the separation of the organic layer, the pH of the aqueous layer was adjusted to 7.0 using concentrated hydrochloric acid (65 Kg). The resulting suspension was stirred at 10° C. for 2 hours and filtered. The solids were washed with water (210 Kg) and dried at 70° C. with a nitrogen gas sweep until the water content was below 0.1% to obtain 77 Kg of the crude product (compound of Formula C). The crude solid was suspended in n-butanol (1656 Kg) and heated to 108° C. for dissolution. The solution was slowly cooled to 0 to 5° C. over 7-8 hours and stirred at this temperature for 6 h. The solids were filtered and washed with n-butanol (245 Kg) and acetonitrile (236 Kg). The wet cake was dried at 70° C. with a nitrogen gas sweep until the residual n-butanol was 2700 ppm and residual acetonitrile was 500 ppm to obtain compound of the Formula C as a white solid (69 Kg, 71% yield, HPLC purity=99.8% area).

The product was analyzed by LCMS (Cortecs C18+, 90Å, 2.7 μm, 2.1 mm×30 mm, 3 min method, 0.1% Formic acid, 5-100% MeCN/water): m/z 230.1 (M+H)⁺ (ES⁺), at 0.84 min, 99% purity at 260 nm +/−80 nm.

¹H NMR (400 MHZ, DMSO-d₆) δ 11.10 (br s, 1H), 7.81 (s, 1H), 6.66 (br s, 1H), 3.93-3.77 (m, 3H), 3.36 (td, J=11.5, 2.2 Hz, 2H), 1.87-1.77 (m, 2H), 1.51-1.37 (m, 2H).

¹³C NMR (101 MHz, DMSO) δ 158.04 (C), 153.27 (CH+C), 108.44 (C), 65.72 (2×CH₂), 46.69 (CH), 32.29 (2×CH₂).

### Example 2

### Preparation of 4,5-dichloro-N-(oxan-4-yl)pyrimidin-2-amine, compound of Formula (D)

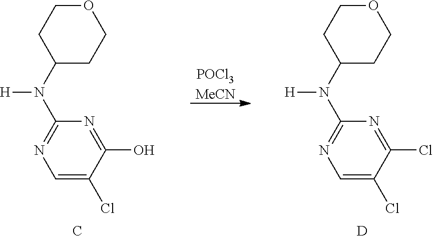

Phosphoryl chloride (185 Kg, 4 eq) was added to a mixture of compound of Formula C (69 Kg, 1 eq) in acetonitrile (550 Kg) and heated to 70 to 75° C. for 6 hours. After the reaction completion, the reaction mixture was cooled to about 35° C. and concentrated to 3-4 volumes. Acetonitrile (270 Kg) was charged followed by concentration to 3-4 volumes. The mixture was cooled to room temperature and added to an aqueous solution of K₃PO₄ (414 Kg) in water (1662 Kg). The resulting suspension stirred at 20 to 25° C. for 2 hours, filtered and washed with pre-heated (30 to 40° C.) water (345 Kg). The wet cake was dried at 45 to 50° C. with a nitrogen sweep for 3 days to obtain compound of Formula D as a white solid (68.6 Kg, 90% yield, HPLC purity=99.9% area).

The product was analyzed by LCMS (Cortecs C18+, 90Å, 2.7 μm, 2.1 mm×30 mm, 3 min method, 0.1% Formic acid, 5-100% MeCN/water): m/z 248.0/250.0 (M+H)⁺ (ES⁺), at 1.69 min, 99% purity at 260 nm +/−80 nm.

¹H NMR (400 MHZ, DMSO-d₆) δ 8.41 (s, 1H), 7.93 (d, J=7.7 Hz, IH), 3.95-3.72 (m, 3H), 3.45-3.27 (m, 2H), 1.85-1.71 (m, 2H), 1.56-1.39 (m, 2H).

¹³C NMR (101 MHz, DMSO) δ 159.50 (C), 158.10 (CH), 156.61 (C), 113.70 (C), 65.89 (2×CH₂), 47.19 (CH), 32.05 (2×CH₂).

### Example 3

### Preparation of tert-butyl (2R)-2-(6-(5-chloro-2-[(oxan-4-yl)amino]pyrimidin-4-yl)-1-oxo-1,3-dihydro-2H-isoindol-2yl)propanoate, compound of Formula (N)

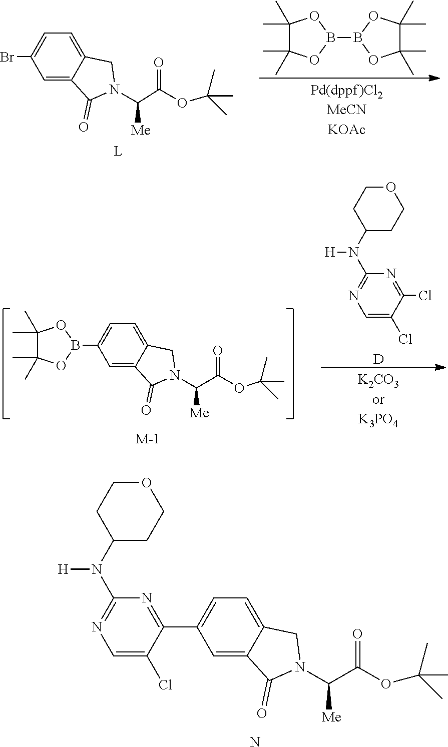

A mixture of compound of Formula L (91.8 Kg, 1 eq), bis(pinacolato)diboron (82 Kg, 1.2 eq), potassium acetate (79 Kg, 2.95 eq) and Pd(dppf)Cl₂ (5 Kg, 0.025 eq) in acetonitrile (734 Kg) was heated to 80 to 85° C. for 2 hours. After conversion of compound of Formula L to compound of Formula M-1, the reaction mixture was cooled to 70 to 75° C. and compound of Formula D (68 Kg) and an aqueous solution of K₃PO₄ (143 Kg) in water (789 Kg) were added. The reaction mixture was heated at 70-75° C. for 48 hours until compound of Formula M-1 was less than 1% and then cooled to room temperature. To the reaction mixture was charged, an aqueous solution of sodium chloride (186 Kg) in water (733 Kg) and ethyl acetate (1656 Kg). The aqueous layer was separated, and the organic layer was concentrated to 8 to 10 volumes. Ethyl acetate (1656 Kg) was added and distilled down to around 9 volumes. Fresh ethyl acetate (1656 Kg) was added and the organic layer was washed with an aqueous solution of citric acid (46 Kg) in water (882 Kg) followed by four washes of 2% aqueous solution of N-acetyl cysteine (10 volumes each wash). The organic layer was washed with 5% aqueous K₂HPO₄ (10 volumes) followed by treatment of the organic layer with activated carbon (18 Kg). The organic layer was filtered through a pad of silica gel (18 cm in height: 200 Kg) and washed with ethyl acetate (4000 Kg). The filtrate was concentrated to about 5-6 volumes and swapped with acetonitrile (825 Kg: 2 times) concentrating to 5-6 volumes each time. The acetonitrile solution was heated to 55-60° C. and water (1560 Kg) was added over 5 hours. The mixture was cooled to room temperature over 5 hours and maintained at this temperature for 6 hours. The resulting suspension was filtered, washed with a mixture of acetonitrile (367 Kg) and water (1100 Kg). The wet cake was dissolved in acetonitrile (1190 Kg) at 58 to 62° C. and water (1530 Kg) was added over 5 hours at that temperature. The mixture was cooled to room temperature over 5 h and stirred at this temperature for 3 hours. The resulting suspension was filtered, washed with a mixture of acetonitrile (367 Kg) and water (1100 Kg). The wet cake was dried at 35 to 45° C. under vacuum until residual water was less than 0.1% to obtain compound of the Formula N as a white solid (90.6 Kg, 71% yield, HPLC purity=99.5% area).

The product was analyzed by LCMS (Cortecs C18+, 90Å, 2.7 μm, 2.1 mm×30 mm, 3 min method, 0.1% Formic acid, 5-100% MeCN/water): m/z 473.2/475.2 (M+H)⁺ (ES⁺), at 2.04 min, 99% purity at 260 nm +/−80 nm.

¹H NMR (400 MHZ, DMSO-d₆) δ 8.44 (s, 1H), 8.03 (d, J=1.6 Hz, 1H), 7.99 (dd, J=7.9, 1.7 Hz, 1H), 7.76 (d, J=7.9 Hz, 1H), 7.68-7.52 (m, 1H), 4.81 (q, J=7.4 Hz, 1H), 4.63 (d, J=17.7 Hz, 1H), 4.56 (d, J=17.8 Hz, 1H), 3.99-3.80 (m, 3H), 3.44-3.33 (m, 2H), 1.90-1.78 (m, 2H), 1.58-1.47 (m, 5H), 1.40 (s, 9H).

¹³C NMR (101 MHz, DMSO) δ 170.57 (C), 167.06 (C), 160.01 (C), 158.22 (CH), 143.61 (C), 136.10 (C), 132.24 (CH), 131.73 (C), 123.54 (CH), 123.25 (CH), 114.68 (C), 81.31 (C), 66.00 (2×CH₂), 50.00 (CH), 47.07 (CH), 46.99 (CH₂), 32.28 (2×CH₂), 27.58 (3×CH₃), 15.33 (CH₃).

### Example 4

### Preparation of (2R)-2-(6-(5-chloro-2-[(oxan-4-yl)amino]pyrimidin-4-yl)-1-oxo-1,3-dihydro-2H-isoindol-2yl)propanoic acid, compound of Formula (O)

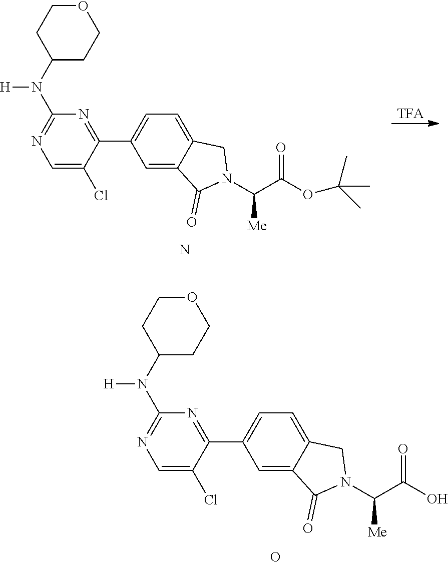

To a solution of compound of Formula N (11.8 Kg, 1 eq) in dichloromethane (312 Kg) was added trifluoroacetic acid (36 Kg, 13 eq). The reaction mixture was stirred at 20 to 30° C. for 35 hours until compound of Formula N is consumed to below 1%. The reaction mixture was distilled down to 4 volumes and swapped with toluene (3×123 Kg) distilling down to 4 volumes. Dichloromethane (126 Kg) was added and the organic layer was washed with 10% aqueous solution of K₂HPO₄ (2×177 Kg).

The combined aqueous layer was washed once with dichloromethane (82 Kg). The aqueous layer was heated to 55 to 62° C. and the pH was adjusted to 3.0 using aqueous hydrochloric acid. The mixture was cooled to 20 to 25° C. over 3 hours and maintained at this temperature for 2-3 hours. The precipitated solids were filtered, washed with water (2×35 Kg) and dried at 40-45° C. with a flow of nitrogen gas for 12 hours to obtain compound of Formula O as a white solid (9.6 Kg, 90% yield; HPLC purity=100% area).

The product was analyzed by LCMS (Cortecs C18+, 90Å, 2.7 μm, 2.1 mm×30 mm, 3 min method, 0.1% Formic acid, 5-100% MeCN/water): m/z 473.2/475.2 (M+H)⁺ (ES⁺), at 2.04 min, 99% purity at 260 nm +/−80 nm.

¹H NMR (400 MHZ, DMSO-d₆) δ 8.44 (s, 1H), 8.03 (d, J=1.6 Hz, 1H), 7.99 (dd, J=7.9, 1.7 Hz, 1H), 7.76 (d, J=7.9 Hz, 1H), 7.68-7.52 (m, 1H), 4.81 (q, J=7.4 Hz, 1H), 4.63 (d, J=17.7 Hz, 1H), 4.56 (d, J=17.8 Hz, 1H), 3.99-3.80 (m, 3H), 3.44-3.33 (m, 2H), 1.90-1.78 (m, 2H), 1.58-1.47 (m, 5H), 1.40 (s, 9H).

¹³C NMR (101 MHz, DMSO) δ 170.57 (C), 167.06 (C), 161.5 (broad peak) 160.01 (C), 158.22 (CH), 143.61 (C), 136.10 (C), 132.24 (CH), 131.73 (C), 123.54 (CH), 123.25 (CH), 114.68 (C), 81.31 (C), 66.00 (2×CH₂), 50.00 (CH), 47.07 (CH), 46.99 (CH₂), 32.28 (2×CH₂), 27.58 (3×CH₃), 15.33 (CH₃).

### Example 5

### Preparation of (2R)-2-(6-{5-chloro-2-[(oxan-4-yl)amino]pyrimidin-4-yl}-1-oxo-2,3-dihydro-1H-isoindol-2-yl)-N-[(1S)-1-(3-fluoro-5-methoxyphenyl)-2-hydroxyethyl]propenamide, compound of Formula (I)

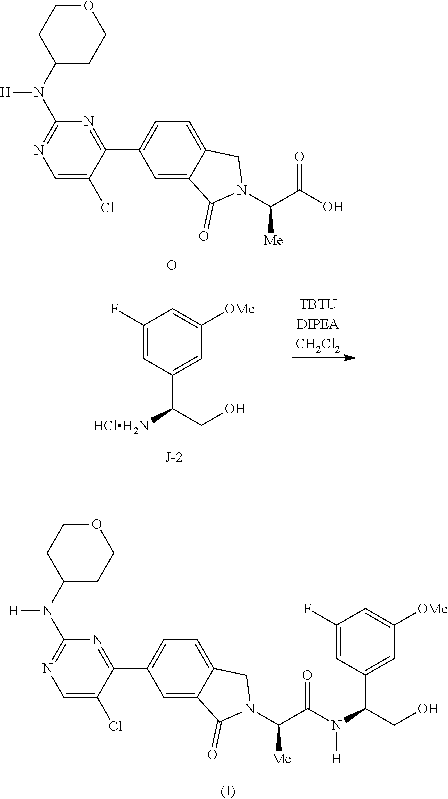

A mixture of compound of Formula O (9.29 Kg. 98.2% assay, 1 eq), compound of Formula J-2 (6.06 Kg. 1.2 eq) and N-ethyldiisopropyl amine (12 Kg, 4 eq) in dichloromethane (247 Kg) was cooled to −3 to 3° C. and TBTU (8.79 Kg, 1.2 eq) was added in five portions with 15 min interval between each portion. The reaction mixture was stirred at this temperature for 1 h and HPLC analysis indicated consumption of compound of Formula O, less than 1%. The reaction mixture was warmed to room temperature and washed two times with 10% aqueous hydrochloric acid (95 Kg) followed by two times with 10% aqueous K₂HPO₄ (95 Kg). The organic layer washed with water (95 Kg) and distilled down to 5-6 volumes. Dichloromethane was swapped with anhydrous ethanol (143 Kg) and distilled down to 6 volumes. The ethanolic solution was heated to about 50° C. and water (67 Kg) was added over 2 hours. The mixture was seeded with compound of Formula I (80 g) and stirred at this temperature for 10 hours. The resulting suspension was cooled to room temperature over 5 hours and stirred at this temperature for 3 hours, filtered and washed with a mixture of ethanol (15 Kg) and water (19 Kg). The wet cake was dried at 30 to 35° C. with a stream of nitrogen gas until residual water was less than 4% to obtain crude compound of the Formula I (11.2 Kg). The crude product was dissolved in anhydrous ethanol (37 Kg) at about 50° C. and water (22 Kg) was added over 1 h. Seeds of compound of Formula (40 g) were added and water (22 Kg) was added over 1 h. The mixture was stirred at about 50° C. for about 2 hours and cooled to about 40° C. over 1 h. The mixture was stirred at this temperature for 15 hours and cooled to room temperature over 5 hours. After stirring for 2 hours at room temperature, the resulting suspension was filtered and washed with a mixture of ethanol (16 Kg) and water (20 Kg). The wet cake was dried at about 35° C. with a stream of nitrogen over 3 days until the water content was below 3%, to obtain compound of the Formula I as a white solid (10.3 Kg, 77% yield: HPLC purity=99.5% area, chiral purity=100% area).

The product was analyzed by LCMS (Cortecs C18+, 90Å, 2.7 μm, 2.1 mm×30 mm, 3 min method, 0.1% Formic acid, 5-100% MeCN/water): m/z 186.2 (M+H)⁺ (ES⁺), at 0.10 min, 99% purity at 260 nm +/−80 nm. The amount of compound of Formula (Z-1) in the product is about 0.03% area by HPLC, and the amount of compound of Formula (Z-2) in the product is <0.02% area by HPLC.

¹H NMR (400 MHZ, DMSO-d₆) δ 8.69 (s, 3H), 7.03 (t, J=1.9 Hz, 1H), 7.02-6.96 (m, 1H), 6.83 (dt, J=11.0. 2.3 Hz, 1H), 5.54 (t, J=5.1 Hz, 1H), 4.25 (t. J=5.9 Hz, 1H), 3.78 (s, 3H), 3.72 (t, J=5.2 Hz. 2H).

¹³C NMR (101 MHZ, DMSO-d₆) δ 162.79 (C, J=242.6 Hz), 160.71 (C, J=11.7 Hz), 139.05 (C, J=9.9 Hz), 110.16 (CH, J=2.6 Hz), 106.65 (CH, J=22.9 Hz), 101.30 (CH, J=25.0 Hz), 62.70 (CH₂), 55.83 (CH₃), 55.59 (CH, J=2.1 Hz).

¹⁹F NMR (376 MHz, DMSO) δ8 −111.31.

### Example 6

### Preparation of tert-butyl (2R)-2-(6-bromo-1-oxo-1,3-dihydro-2H-isoindol-2-yl)propanoate, compound of Formula (L)

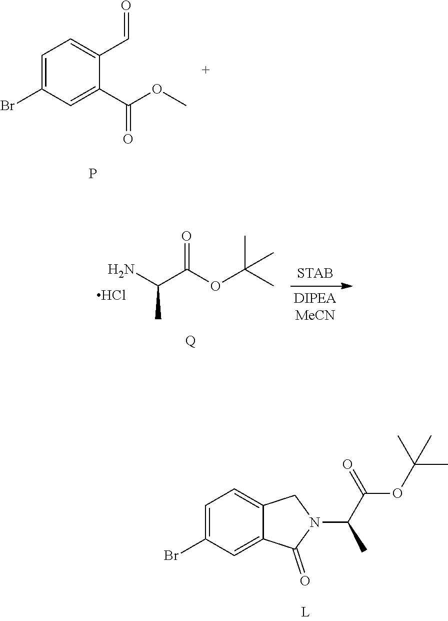

A mixture of tert-butyl D-alaninate, HCl salt (859 mg, 97% Wt, 1.15 Eq, 4.59 mmol), methyl 5-bromo-2-formylbenzoate (1.00 g, 97% Wt, 1 Eq, 3.99 mmol) and DIPEA (1.05 mL, 1.5 Eq, 5.99 mmol) was stirred in benzotrifluoride (10 mL) at room temperature for 1 hour, then was concentrated in vacuo and azeotroped with MeCN (20 mL). The resulting residue was re-dissolved in benzotrifluoride (10 mL) and sodium triacetoxyborohydride (2.11 g, 2.5 Eq, 9.98 mmol) was added in portions over 15 mins. The reaction was stirred for 2 hours at room temperature, then was quenched with water (20 mL). The aqueous layer was extracted with a further portion of benzotrifluoride (10 mL) and the combined organic layers were washed with HCl (10 mL, IM aqueous) and NaHCO₃ (10 mL, saturated aqueous). The organic phase was concentrated to ˜2 mL and the temperature was maintained at 70° C. while n-heptane (7 mL) was added. The reaction was cooled slowly to 35° C. and crystallization occurred. The reaction was then cooled to 18° C. for 10 mins, then the precipitate was collected. The precipitate was washed on the filter with n-heptane (2 mL), then dried in a vacuum desiccator at 45° C. for 2 h, to give tert-butyl (R)-2-(6-bromo-1-oxoisoindolin-2-yl)propanoate (0.80 g, 2.3 mmol, 58% yield, HPLC purity=99% area) as a crystalline white solid.

The product was analyzed by LCMS (XSelect CSH C18 Column, 130Å, 2.5 μm, 4.6 mm×30 mm, Acidic (0.1% Formic acid), 4 min method, 5-95% MeCN/water): m/z 284.0/286.0 (M-tBu)⁺ (ES⁺), at 2.22 min, 99% purity at (diode array).

¹H NMR (400 MHz, DMSO-d₆) δ 7.90-7.78 (m, 2H), 7.61 (dd, J=8.0, 0.8 Hz, 1H), 4.77 (q, J=7.4 Hz, 1H), 4.53 (d, J=17.7 Hz, 1H), 4.46 (d, J=17.7 Hz, IH), 1.48 (d, J=7.5 Hz, 3H), 1.38 (s, 9H).

### Example 7

### Preparation of ethyl 2-(3-fluoro-5-methoxyphenyl)-2-oxoacetate, compound of Formula (E)

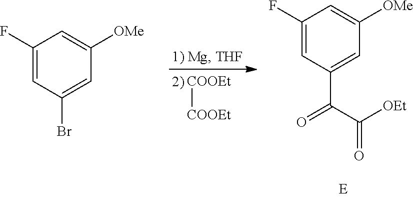 [SMILES: COc1cc(F)cc(Br)c1 ; CCOC(=O)C(=O)OCC ; CCOC(=O)C(=O)c1cc(F)cc(OC)c1]

A slurry of Mg metal (11.9 g, 1.0 eq.) in tetrahydrofuran (200 mL) was heated to 65±5° C. and DIBAL-H (3.6 mL, 0.0073 eq.) and a solution of 3-bromo-5-fluoroanisole tetrahydrofuran (200 mL) were added. The reaction mixture was stirred at 65±5° C. until the consumption of 3-bromo-5-fluoroanisole by TLC. The resulting solution was cooled to 5±5° C. and added dropwise to a solution of diethyl oxalate (71.7 g, 1.2 eq.) in tetrahydrofuran (1 L) maintained at −25±5° C. The reaction mixture was stirred at this temperature for one hour and was warmed to 25±5° C. and stirred at 25±5° C. for one hour. The reaction was quenched by the addition of saturated aqueous NH₄Cl solution (500 mL) and then extracted with ethyl acetate (3×100 mL). The combined organic layer was washed with brine (500 mL) and then concentrated to dryness to obtain compound of the Formula E as a yellow liquid (116.8 g, yield=66%, HPLC purity=55.8% area).

### Example 8

### Preparation of ethyl 2-(3-fluoro-5-methoxyphenyl)-2-oxoacetate, compound of Formula (E)

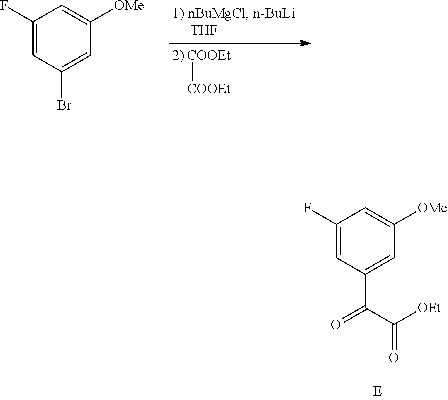 [SMILES: COc1cc(F)cc(Br)c1 ; CCOC(=O)C(=O)OCC ; CCOC(=O)C(=O)c1cc(F)cc(OC)c1]

To a solution of 3-bromo-5-fluoroanisole (550 g, 1 eq) in tetrahydrofuran (5.5 L) was cooled to −10° C. and nBuMgCl (0.33 eq) and nBuLi (0.67 eq) were added slowly. The reaction mixture was stirred at this temperature until the consumption of the starting material (by HPLC) and then added dropwise to a solution of diethyl oxalate (3 eq) in tetrahydrofuran (2.75 L) maintained at −55° C. The reaction mixture was maintained at this temperature until the consumption of starting material followed by quenching with saturated aqueous ammonium chloride (2.75 L). The product was extracted into methyl tert-butylether (2×2.75 L) and the combined organic layer was washed with brine (2.75 L) and concentrated to dryness to obtain compound of Formula E as orange oil (610 g, yield=71%, HPLC purity=73.79% area).

### Example 9

### Preparation of compound of Formula (G-1)

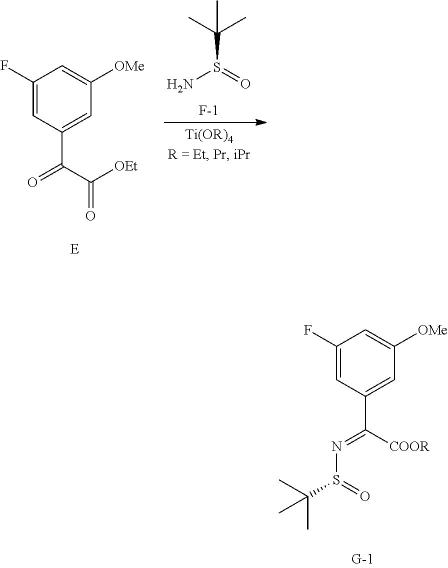

To a solution of compound of Formula E (600 g, 1 eq) in tetrahydrofuran (6 L) was charged R-(+)-2-methyl-2-propanesulfinamide (385 g, 1.2 eq.) and Ti(OEt)₄ (1391 g, 2.3 eq). The mixture was heated to about 60° C. until reaction completion, then cooled to 50° C. and EDTE (N,N,N′,N′-tetrakis(2-hydroxyethyl)ethylenediamine) (1567 g, 2.5 eq) was charged. The reaction mixture was stirred at 50° C. for 30 min and cooled to room temperature. Methyl tert-butylether (3 L) and water (3 L) were charged, stirred for 30 min and phases separated. The aqueous layer was extracted once with methyl tert-butylether (3 L) and the combined organic layer was washed with brine (3 L) and concentrated to dryness to afford compound of Formula G-1 as a brown oil (1187 g, yield=84%; HPLC purity: ethyl ester=68.75% area and isopropyl ester=17.83% area).

### Example 10

### Preparation of (S)-2-amino-2-(3-fluoro-5-methoxyphenyl)ethan-1-ol, hydrogen chloride salt, compound of Formula (J-2)

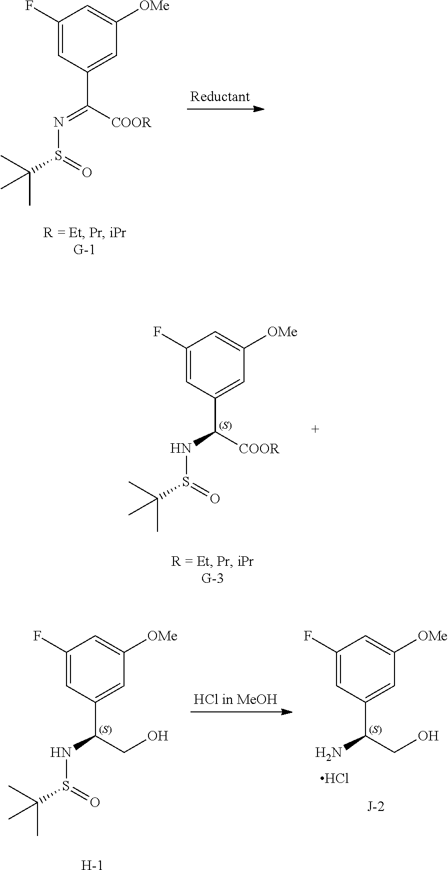

A solution of compound of Formula G-1 (449 g, 1 eq) in tetrahydrofuran (4.49 L) was cooled to −35° C. and a solution of borane (1M in tetrahydrofuran, 3.0 eq) was added dropwise maintaining the temperature between −30 to −40° C. The reaction mixture was stirred at −35° C. for 2 hours and upon completion of reaction, the mixture was warmed to room temperature. The reaction mixture was stirred at room temperature for 16 hours until the conversion of compound of Formula G-3 to compound of Formula H-1 is complete. The reaction mixture was then added dropwise to methanol (2.245 L) cooled to 5° C. caution: hydrogen gas evolution. A solution of hydrochloric acid (4M in methanol, 6 eq) was added dropwise at 5° C. The reaction mixture was warmed to room temperature and stirred at this temperature for 16 hours until the conversion of compound of Formula H-1 to compound of Formula J-2 is complete. The reaction mixture was concentrated under vacuum to 5 volumes and the solvent was exchanged with methyl tert-butylether (2×4.5 L) until residual methanol was less than 1%. The 5 volume methyl tert-butylether solution was stirred at room temperature for 1 hour and the resulting suspension was filtered, washed with methyl tert-butylether (900 mL). The wet was dried under vacuum to obtain compound of the Formula J-2 as a white solid (390.3 g, yield=75%, HPLC purity=96.3% area, chiral purity: 91.5% area).

### Example 11

### Preparation of (S)-2-amino-2-(3-fluoro-5-methoxyphenyl)ethan-1-ol, compound of Formula (J-1)

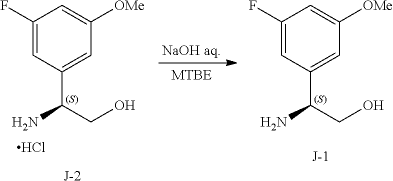

To a slurry of crude compound of Formula J-2 (748 g, 1 eq) in methyl tert-butyl ether (7.48 L) at 5° C. was added slowly an aqueous solution of sodium hydroxide (1.7 M, 2.992 L). After the addition, the mixture was brought to room temperature and stirred for 1 hour. The phases were separated and the aqueous layer was extracted with methyl tert-butyl ether (2×7.48 L). The combined organic layer was washed with brine (3.74 L) and concentrated to dryness. The residual water was removed to less than 3% using azeotropic distillation to dryness with methyl tert-butyl ether to obtain compound of Formula J-1 as light yellow solid (563 g, yield=93%, HPLC purity=96.74% area, chiral purity=94.93% area).

### Example 12

### Preparation of (S)-2-amino-2-(3-fluoro-5-methoxyphenyl)ethan-1-ol L-mandelic acid salt, compound of Formula (J-3)

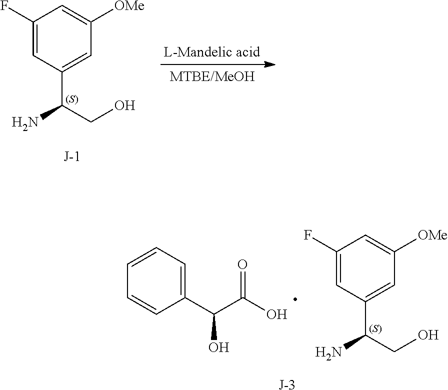

To a solution of compound of Formula J-1 (333 g, 1 eq) in methyl tert-butyl ether (3.33 L) at room temperature, was charged (S)-(+)-mandelic acid (274 g, 1 eq). The resulting suspension was stirred at room temperature for 1 hour and methanol (500 mL) was charged. The reaction mixture was warmed to 45° C. and stirred at this temperature for 16 hours. The reaction mixture was cooled to room temperature and stirred for 3 hours. The solids were filtered, washed with methyl tert-butyl ether (1.33 L) and dried under vacuum to obtain mandelate salt of Formula J-3 as a white solid (520 g, yield=86%, HPLC purity=99.1% area, chiral purity=99.8% area).

### Example 13

### Preparation of (S)-2-amino-2-(3-fluoro-5-methoxyphenyl)ethan-1-ol (R)-tetrahydrofuran-2-carboxylic acid salt, compound of Formula (J-4)

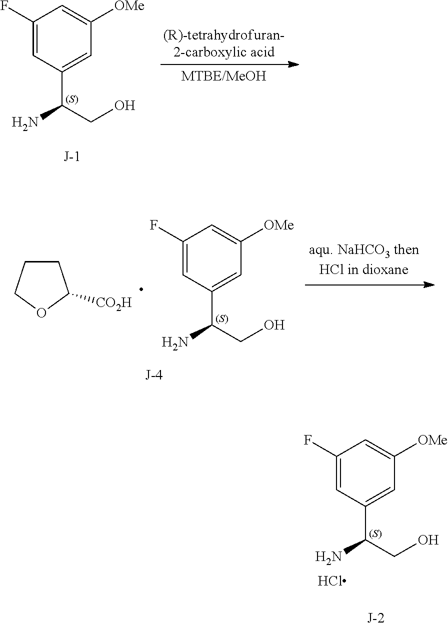

To a solution of compound of the Formula J-1 (630 mg, 1 eq, 3.40 mmol, chiral HPLC=96.68% area) in acetonitrile (113 mL) was added (R)-tetrahydrofuran-2-carboxylic acid (395 mg, 324 μL, 1.00 Eq, 3.40 mmol). The resulting white suspension was heated at reflux (90° C. external temperature) for 30 minutes to obtain a light yellow solution. The mixture was stirred at this temperature for 1 hour and cooled to room temperature and stirred at this temperature for 12 hours. The solids were filtered, washed with acetonitrile (5 mL) and dried under vacuum to obtain the tetrafuroate salt of Formula (J-4) as a white solid (775 mg 76% yield, chiral HPLC=100% area).

The compound of Formula (J-4) (775 mg, 1 eq) was partitioned between dichloromethane (20 mL) and saturated aqueous sodium bicarbonate solution (20 mL). The aqueous layer was extracted with dichloromethane (2×10 mL) and the combined organic layer was dried over anhydrous sodium sulphate, filtered and concentrated to about 5 mL. A solution of hydrochloric acid (4 N in dioxane, 3 eq, 1.9 mL) was added. The mixture was stirred for 30 min and evaporated to dryness to obtain compound of the Formula J-2 as a white solid (523 g, chiral purity=100% area)

### Example 14

### Preparation of (S)-2-amino-2-(3-fluoro-5-methoxyphenyl)ethan-1-ol, compound of Formula (J-1)

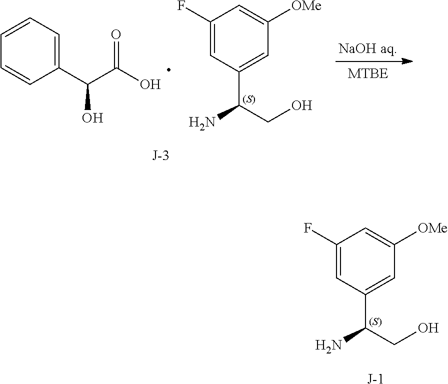

To a solution of compound of Formula J-4 (mandalate salt) (515 g, 1 eq) in methyl tert-butyl ether (5.15 L) at 5° C., was added aqueous solution of sodium hydroxide (0.46 M, 2.575 L, 1.5 eq). The mixture was warmed to room temperature and stirred for 2 hours. The phases were separated and the aqueous layer was extracted with methyl tert-butyl ether (3×2.575 L). The combined organic layer was concentrated under vacuum to 1 volume which contained 259 g of compound of Formula J-1 (yield=92%, HPLC purity=99.62% area, chiral purity=99.9% area).

### Example 15

### Preparation of (S)-2-amino-2-(3-fluoro-5-methoxyphenyl)ethan-1-ol hydrogen chloride salt, compound of Formula (J-2)

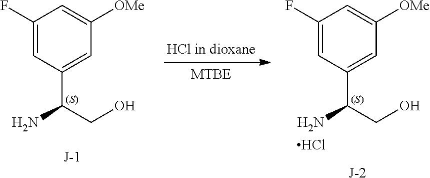

To a solution of compound of Formula J-1 (259 g, 1 eq) in methyl tert-butyl ether (2.5 L) at room temperature, was added hydrochloric acid (4 M in dioxane, 763 mL, 2 eq). The mixture was stirred at room temperature for 3 hours and the resulting solids were filtered, washed with methyl tert-butyl ether (1.56 L). The wet cake was dried under vacuum to obtain compound of the Formula J-2 as a white solid (306 g, yield=99%, HPLC purity=99.8% area, chiral purity=99.89% area, residual L-mandelic acid<100 ppm).

### Example 16

### Preparation of compound of Formula (R)

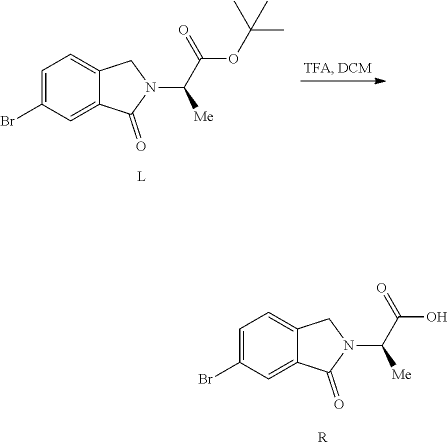

Trifluoroacetic acid (43.5 g, 381.8 mmol, 13 eq.) was added over 15 minutes to a solution of Compound of the Formula L (10 g, 29.4 mmol, 1.0 eq.) in dichloromethane (200 mL). The reaction mixture was stirred at 35° C. for 18 h. The reaction mixture was concentrated to 3 volumes at 30° C. and distilled twice with toluene (add 10 volumes of toluene and concentrate down to 3 volumes each time). The resulting slurry was completely concentrated to obtain a white solid which was triturated with ethylacetate (200 mL) at 75° C. for 30 minutes, cooled to 5° C. over 30 minutes, hold for 30 minutes, and filtered. The white solid was dried under high vacuum for 4h to obtain the product (7.5 g, yield=90%) as a white solid.

### Example 17

### Preparation of Compound of Formula (T)

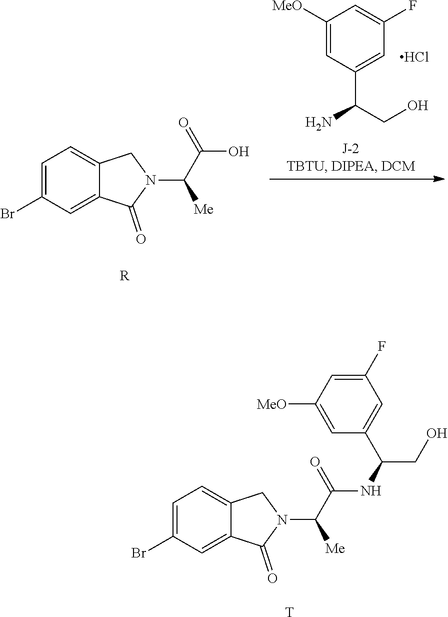

A solution of carboxylic acid compound R (6 g, 21.1 mmol, 1.0 eq.), compound of Formula J-1 (5.6 g, 25.3 mmol, 1.2 eq.) and N-ethyldiisopropylamine (10.9 g, 84.4 mmol, 4.0 eq.) in dichloromethane (72 mL) was cooled to 0° C. TBTU (8.4 g, 25.3 mmol, 1.2 eq.) was added in five portions over calculated 70 minutes and the reaction mixture was stirred at 0° C. for 3 hours. The reaction mixture was successively washed with 1N HCl (2×60 mL), 10% K₂HPO₄ solution (2×60 mL) and water (60 mL). The organic layer was filtered over a short pad of anhydrous Na₂SO₄ and completely concentrated to obtain an off-white solid. The crude solid was dissolved in ethanol (60 mL) at 75° C., cooled to 50° C. over 1 hour to obtain a spongy slurry. Water (60 mL) was added over 1 h, cooled to 20° C. over 30 minutes, and stirred for 1 h. The slurry was then filtered and rinsed with a mixture of ethanol/water (1:1, 12 mL×2) to obtain white cotton like solid. The wet solid was dried under vacuum at 35° C. for 20 hours to obtain amide compound T (7.5 g, 79% yield) as white fluffy solid.

### Example 18

### Preparation of Compound of Formula (I)

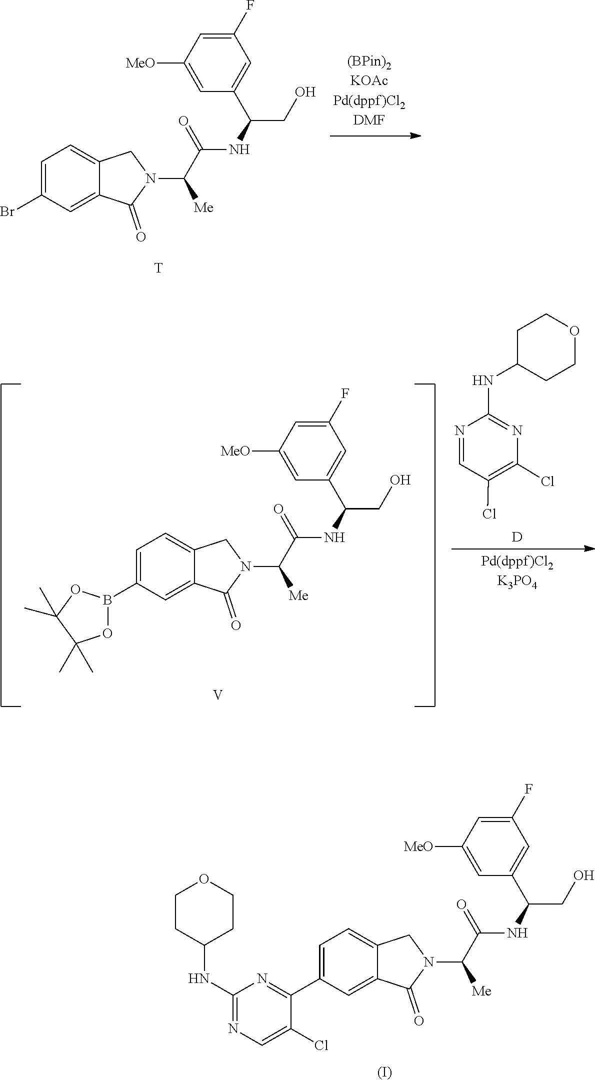

To a dry flask under nitrogen was added DMF (30 mL), compound T (3.0 g, 6.64 mmol, 1.0 eq), Bis-pinacolatodiboron (2.0 g, 7.9 mmol, 1.2 eq.), KOAc (1.92 g, 19.6 mmol, 2.95 eq.) and Pd(dppf)Cl₂ (0.122 g, 0.166 mmol, 0.025 eq.). The reaction mixture was degassed (evacuate to 200 mbar and refill with nitrogen, 3 times). The reaction mixture was heated to 110° C. over 30 minutes and stirred for 4h (TLC in EtOAc showed very faint spot for amide left). The reaction mixture was then diluted with EtOAc (100 mL) and phases separated. The aqueous layer was further extracted with EtOAc (50 mL). The combined organic layer was washed with water (50 mL) and brine (50 mL) and filtered via a pad of anhydrous Na2SO4 (2 cm) and celite (1 cm). The filtrate was concentrated to obtain a dark oil which was purified via column chromatography (0% to 10% MeOH in DCM) to obtain crude boronate compound V (3.4 g) as dark paste. The crude boronate (3.4 g, presumed 6.64 mmol, 1.0 eq.) was dissolved in DMF (30 mL) and Pd(dppf)Cl₂ (0.122 g, 0.166 mmol, 0.025 eq.) was added followed by the addition of compound of Formula D (1.65 g, 6.64 mmol, 1.0 eq.) and K₃PO₄ aqueous solution (3.52 g, 16.6 mmol, 2.5 eq in 30 mL water). The reaction mixture was then stirred at 80° C. for 12 hours (TLC showed consumption of compound of Compound D). The reaction was quenched by transferring reaction mixture to 20% NaCl solution (30 mL) and EtOAc (60 mL) was added, and phases were separated. The aqueous layer was extracted with EtOAc (30 mL). The combined organic layer was then successively washed with 5% citric acid solution (30 mL×2), 5% K₂HPO₄ solution (30 mL×2) and water (30 mL). The organic layer was then concentrated and purified by column chromatography to obtain partially pure fractions which were concentrated to obtain compound of Formula I as a brown foam (2.9 g, HPLC purity=84.5%). The crude product was then recrystallized from EtOH/H₂O (1:1) to obtain compound of the Formula (I) as a pale-yellow solid (2.3 g, yield=59%, HPLC purity=97% area).

### Example 19

### Preparation of a Compound of Formula (J-2)

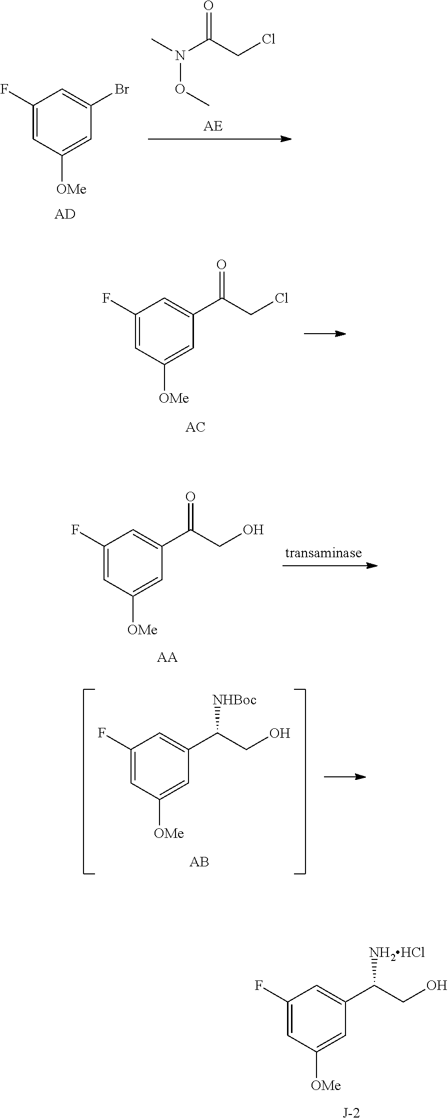

### Step 1: Preparation of Compound of Formula (AC)

To a solution of 3-fluoro-5-bromoanisole (1.0 Kg, 1.0 eq) in tetrahydrofuran (3 L) at-10° C., was added slowly a 1.3 M solution of iPrMgCl.LiCl (5.63 L, 1.5 eq) in tetrahydrofuran, maintaining temperature between −15 to −5° C. The reaction mixture was warmed to 20-25° C. and stirred at this temperature for 4 hours. HPLC analysis indicated 0.51% of unreacted starting material. The reaction mixture was cooled to −55 to −45° C. and a solution of 2-chloro-N-methoxy-N-methylacetamide (compound of Formula (AE) (805 g, 1.2 eq) in tetrahydrofuran (4 L) was added slowly maintaining the temperature between −55 to −45° C. The reaction mixture was warmed to 15 to 25° C. and stirred at this temperature for 14 hours. The reaction mixture was cooled to −10 to 0° C. and quenched with IM hydrochloric acid (6 L). The organic layer was separated, and the aqueous layer was extracted with ethyl acetate (5 L). The combined organic layer was washed with saturated sodium chloride solution (10 L) and concentrated to about 5 L remaining in the reactor. Ethyl acetate (2 L×3) was added and concentrated under vacuum to about 5 L. N-Heptane (4 L) was added over 2 hours and the reaction mixture was stirred at 15 to 25° C. for 2 hours. The resulting solids were filtered, washed with n-heptane (2 L) and dried under vacuum at 40° C. to obtain compound of the Formula (AC) (760 g, yield: 70%, HPLC purity: 98.15% area).

### Step 2: Preparation of Compound of Formula (AA)

To a solution of compound of Formula (AC) (1.328 Kg, 1 eq) in anhydrous ethanol (6.38 L) was added water (10.63 L), formic acid (1.207 Kg, 4 eq) and sodium formate (1.471 Kg, 3.3 eq). The reaction mixture was heated to 85 to 95° C. and stirred at this temperature for 8 hours. HPLC analysis indicated 0.61% of unreacted starting material (compound of Formula (AC)). The reaction mixture was cooled to 20-25° C. and extracted with methyl tert-butyl ether (13.28 L). The aqueous layer was separated and extracted with methyl tert-butyl ether (6.64 L). The combined organic layer was washed with a solution of sodium carbonate (521 g, 0.75 eq) in water (6.64 L) followed by saturated sodium chloride solution (6.64 L). The organic layer was concentrated under vacuum at 40° C. to about 4000 L remaining in the reactor. Anhydrous ethanol (5.31 L) was charged and concentrated to about 4 L remaining in the reactor. N-Heptane (13.28 L) was added over 5-6 hours and the resulting slurry was stirred at 20-25° C. for 16 hours. The slurry was concentrated to 13.28 L remaining in the reactor and n-heptane (6.64 L) was added. The slurry was concentrated to 13.28 L remaining in the reactor and n-heptane (6.64 L) was added. The slurry was concentrated to 13.28 L remaining in the reactor. GC analysis indicated non-detectable levels of residual ethanol. The mixture was filtered and dried under a flow of nitrogen to obtain the product (986 g, yield: 80%, HPLC purity: 96.04%).

### Step 3: Preparation of Compound of Formula (J-2)

To compound of Formula (AA) (10 g) was added EW-TA-184 (2 g, 20 w/w %: purchased from Enzyme Works Inc., Zhangjiagang, Jiangsu, China) a 0.1 M solution of tris(hydroxymethyl)amino methane (500 mL), isopropylamine (20 eq. 66 mL), dimethyl sulfoxide (750 mL) and pyridoxal phosphate (2 g). The reaction mixture with a pH of 9.0 was stirred at 20 to 35° C. for 4 hours. HPLC analysis indicated a 98.3% reaction conversion. Solid sodium hydroxide (42 g) was added to adjust the pH to 13.16. The mixture was stirred for 1 hour and filtered. The mixture was concentrated under reduced pressure below 40° C. for about 2 hours. Ditert-butyl decarbonate (2 eq) was added and the reaction mixture was stirred at 20-25° C. for 20 hours. The reaction mixture was extracted three times with dichloromethane (200 mL each time). The combined organic layer was washed with water (3×200 mL) and saturated sodium chloride solution (3×200 mL). The organic layer was concentrated under reduced pressure to about 15 mL remaining in the reactor, and n-heptane (300 mL) was added over 30 min. The slurry was stirred at 20-25° C. for 1 hour, filtered and washed with n-heptane (2×20 mL). HPLC analysis of the wet cake showed a purity of 98.98% area of boc-protected compound of Formula (AB). The damp cake was dissolved in dichloromethane (100 mL) and 4M hydrochloric acid in 1,4-dioxane (4.0 eq) was added. The reaction mixture was stirred at 20-25° C. for 15 hours, filtered, washed with dichloromethane (20 mL) and dried under vacuum to obtain the compound of Formula (J-2) (7.16 g, yield: 60%, HPLC purity: 99.5% area, chiral purity: 100% area)

### Example 20

### Preparation of a Compound of Formula (J-2)

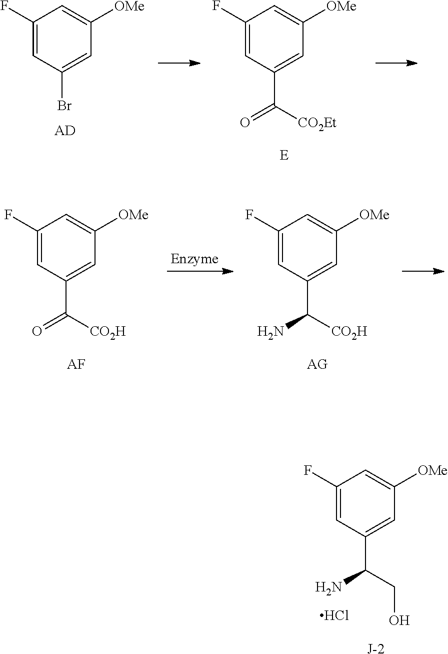

### Step 1: Preparation of Compound of Formula (E)

To a mixture of magnesium metal (15.8 Kg) and iodine (0.8 Kg) in tetrahydrofuran (344.1 Kg) was added 3-fluoro-5-bromoanisole (12.8 Kg) at 10-25° C. The mixture was heated to 60-65° C. and stirred at this temperature for 3-5 hours. A solution of 3-fluoro-5-bromoanisole (115.4 Kg, 1 eq) in tetrahydrofuran (342.7 Kg) was added at the rate of 20-35 Kg/hr maintaining a temperature of 55-65° C. The reaction mixture was stirred at 55-65° C. for 3 hours and cooled to 10-25° C. This reaction mixture was added to a solution of diethyl oxalate (95.8 Kg) in tetrahydrofuran (911 Kg) at −75 to −65° C. at the rate of 60-150 Kg/hr. The reaction mixture was stirred at −75 to −65° C. for 6.5 hours until reaction completion. This reaction mixture was quenched into a solution of hydrochloric acid (97.4 Kg) in water (172 Kg) at −20 to 30° C., adding at a reference rate of 100-200 Kg/hr. The mixture was stirred at 20-30° C. for 1 hour and solid sodium chloride (22.2 Kg) was added. The organic layer was separated and concentrated under vacuum at 45° C. until 1-2 volumes left to obtain compound of the Formula (E) (256 Kg, assay: 38.59%, HPLC purity: 68.11% area).

### Step 2: Preparation of Compound of Formula (AF)

To a solution of sodium hydroxide (103.2 Kg) in water (1288 Kg) at 20-30° C. was added the tetrahydrofuran solution of compound of Formula (E) (255.8 Kg, 98.7 Kg corrected for 38.59% assay). The reaction mixture was stirred at 20-30° C. for 11 hours until reaction completion by HPLC analysis. The mixture was filtered, and the cake was washed water (283 Kg). The filtrate was extracted twice with methyl tert-butyl ether (526.9 Kg) and the aqueous layer was acidified with hydrochloric acid at 15-30° C. until a pH of 1 is obtained. The mixture is extracted with methyl tert-butyl ether (617 Kg). The aqueous layer was separated and extracted with methyl tert-butyl ether (621 Kg). The combined organic layer was concentrated under reduced pressure at 40° C. until around 300 L was left, n-Heptane (479.6 Kg) was added and concentrated under vacuum till around 300 L was left, n-Heptane (480.8 Kg) was added and concentrated under vacuum till around 300 L was left. The mixture was heated to 40-45° C., stirred at this temperature for 2 hours and then cooled to 0-5° C. The mixture was stirred at 0-5° C. for 4 hours, and the resulting solids were filtered, washed with cold n-heptane (96.2 Kg) and dried under vacuum at 45° C. to obtain compound of the Formula (AF) (80.4 Kg, assay: 94.58%. HPLC purity: 97% area).

### Step 3: Preparation of Compound of Formula (AG)

A solution of tris(hydroxymethyl)aminomethane (18.0 Kg) in water (1376 Kg) was stirred at 20-25° C. for 30 min and the pH was adjusted to 8.9 using aqueous hydrochloric acid (1:1) (6.0 Kg). A 211 Kg of this solution (solution A) was stored for later use. The compound of Formula (AF) (80.4 Kg. 76 Kg corrected for assay) was charged and the pH was adjusted to 12.4 using 5M sodium hydroxide solution (87.4 Kg). The pH was then adjusted to 9.0 using 6M hydrochloric acid (8.2 Kg). Solution A and ammonium formate (76.4 Kg) were added and the pH was adjusted to 9.1 using 5M sodium hydroxide solution (24.6 Kg). The reaction mixture was maintained between 28-32° C. A solution of FDH enzyme liquid (92.2 Kg) and nicotinamide adenine dinucleotide (1.6 Kg) was prepared and added to the reaction mixture at 28-32° C. At the same temperature. AADH506035 enzyme liquid from Asymchem (33 Kg) was added. The reaction mixture was stirred at 28-32° C. for 15.5 hours and the temperature was adjusted to 15-30° C. 6M hydrochloric acid was charged to adjust the pH to 0.82 and the mixture was filtered in a centrifuge filter equipment rinsing the cake twice with water (240 Kg). The filtrate was extracted twice with methyl tert-butyl ether (300 Kg). The aqueous layer was concentrated under vacuum at 60° C. to around 700 L left in the reactor. The mixture was cooled to 15-25° C. and the pH was adjusted to 5.9 using 50% aqueous potassium carbonate solution. The mixture was cooled to 5-10° C. and stirred at this temperature for 6 h. The mixture was filtered, washed with water (240 Kg) and dried under vacuum at 45-65° C. to obtain the compound of Formula (AG) (yield: 58%, assay: 82.7%. HPLC purity: 99.4% area).

### Step 4: Preparation of a Compound of Formula (J-2)

To a solution of compound of Formula (AG) (53.4 Kg. 44.2 Kg corrected for assay) in tetrahydrofuran (762.6 Kg) cooled to 5-15° C. was added sodium borohydride (25.8 Kg). The reaction mixture was further cooled to −10 to 0° C. and 47% boron trifluoride etherate (164 Kg) was added slowly at a reference rate of 40-70 Kg/h. The mixture was allowed to react at −10 to 0° C. for 18 hours until the consumption of compound of Formula (AG). The reaction mixture was quenched by adding into a solution of hydrochloric acid (66 Kg) in water (595 Kg) at below 10° C. to a pH of 1.4. The reaction mixture was degassed with nitrogen bubbling to remove residual hydrogen gas. Methyl tert-butyl ether (326 Kg) was added and the pH was adjusted to 10.0 using 5M aqueous sodium hydroxide. The mixture was filtered. rinsed with methyl tert-butyl ether (132 Kg). The organic layer in the filtrate was separated and the aqueous layer was extracted twice with methyl tert-butyl ether (326 Kg). The combined organic layer was concentrated under vacuum at 40° C. to around 100 L left in the reactor. Methyl tert-butyl ether (329 Kg) was added and concentrated under vacuum at 40° C. to around 100 L left in the reactor. This process of methyl tert-butyl addition (329 Kg each time) and distillation was repeated for 10 times until a final volume of around 450 L was left in the reactor. The mixture was heated to 50° C. and L-(+)-mandelic acid (33.6 Kg) was added, followed by methanol (24 Kg). The mixture was stirred at 50-60° C. for about 10 hours and then cooled to 20-30° C. The mixture was filtered and rinsed with methyl tert-butyl ether (238 Kg). The wet cake was charged in a reactor and 1M sodium hydroxide (29.6 Kg in 265 Kg of water) added 0-10° C. The mixture was warmed to 20-30° C. and the phases separated. The aqueous layer was extracted three times with methyl tert-butyl ether (133 Kg) and the combined organic layer was washed with a solution of sodium hydroxide (0.2 Kg) and sodium chloride (17.6 Kg) in water (68.4 Kg). The organic phase was concentrated under vacuum at 40° C. to around 150 L left in the reactor. Methyl tert-butyl ether (192 Kg) was added and distilled down to around 150 L left in the reactor. This process of methyl tert-butyl ether (192 Kg each time) addition and distillation was repeated three times. To the concentrate at 20-30° C., 4M hydrochloric acid in 1,4-dioxane (117 Kg) was added at a reference rate of 15-20 Kg/h. The mixture was stirred at 20-30° C. for 4 hours, filtered, washed with methyl tert-butyl ether (90 Kg) and dried under vacuum at 45° C. to obtain compound of Formula (J-2) (39.2 Kg, assay: 99.1%, Yield: 79%, HPLC purity: 99.8% area, chiral HPLC purity: 100% area).

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

What is claimed:

1. A process for preparing a compound of Formula (D), or a salt thereof, 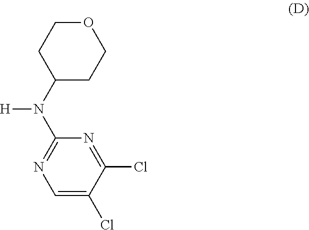 [SMILES: Clc1cnc(NC2CCOCC2)nc1Cl]
comprising:
(i) contacting a compound of Formula (A), or a salt thereof, 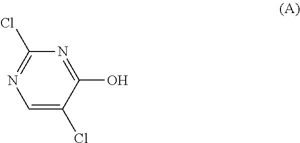
with a compound of Formula (B), or a salt thereof, 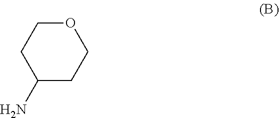
under conditions sufficient to provide a compound of Formula (C), or a salt thereof, 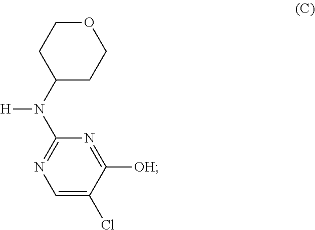 [SMILES: Oc1nc(NC2CCOCC2)ncc1Cl]
and
(ii) chlorinating the compound of Formula (C), or a salt thereof, to provide the compound of Formula (D) or a salt thereof.

2. The process of claim 1, wherein the compound of Formula (A) is a compound of Formula (A-1):

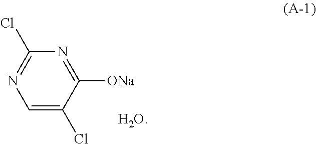

3. The process of claim 1 or claim 2, wherein step (i) further comprises a base and a solvent.

4. The process of claim 3, wherein the base is an amine.

5. The process of claim 4, wherein the amine is diisopropylethylamine.

6. The process of claim 3, wherein the solvent is a protic solvent.

7. The process of claim 6, wherein the protic solvent is n-butanol.

8. The process of any one of claims 1-7, wherein step (ii) is conducted in the presence of phosphoryl chloride.

9. A process for preparing a compound of Formula (J) or a stereoisomer thereof, or a salt thereof, 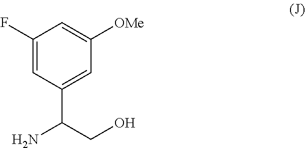 [SMILES: COc1cc(F)cc(C(N)CO)c1]
comprising:
(i) contacting a compound of Formula (E) 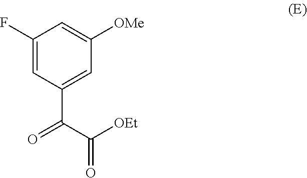 [SMILES: CCOC(=O)C(=O)c1cc(F)cc(OC)c1]
with a compound of Formula (F) or a stereoisomer thereof, or a salt thereof, 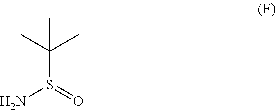
under conditions sufficient to provide a compound of Formula (G) or a stereoisomer thereof, or a salt thereof, 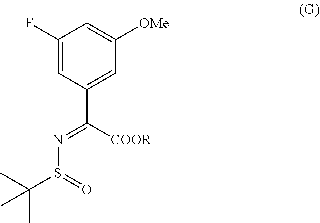
wherein R is C₁₋₅ alkyl; and
(ii) contacting the compound of Formula (G) or a stereoisomer thereof, or a salt thereof, with a reducing agent to provide the compound of Formula (H) or a stereoisomer thereof, or a salt thereof, 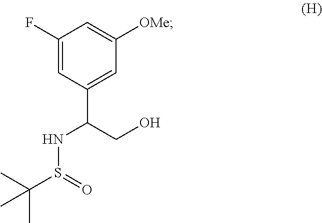 [SMILES: COc1cc(F)cc(C(CO)NS(=O)C(C)(C)C)c1]
and
iii) contacting the compound of Formula (H) or a stereoisomer thereof, or a salt thereof, with an acid to provide the compound of Formula (J) or a stereoisomer thereof, or a salt thereof.

10. The process of claim 9, wherein
the compound of Formula (F) has the structure of Formula (F-1): 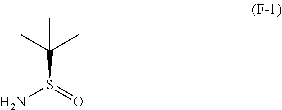
a salt thereof, and
the compound of Formula (G) has the structure of Formula (G-1): 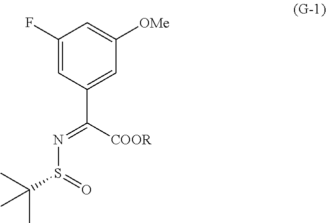
or a salt thereof;
wherein R is C₁₋₅ alkyl;
the compound of Formula (H) has the structure of Formula (H-1): 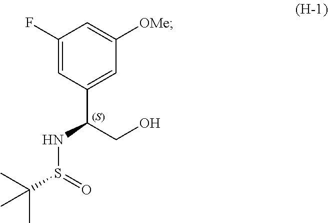
and
the compound of Formula (J) has the structure of Formula (J-1): 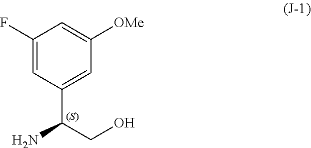
or a salt thereof.

11. A process for preparing a compound of Formula (J) or a stereoisomer thereof, or a salt thereof, 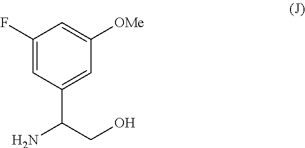 [SMILES: COc1cc(F)cc(C(N)CO)c1]
comprising:
(i) contacting a compound of Formula (E) 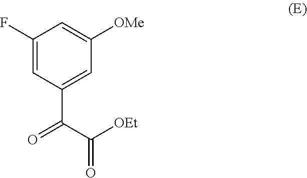 [SMILES: CCOC(=O)C(=O)c1cc(F)cc(OC)c1]
with a compound of Formula (F) or a stereoisomer thereof, or a salt thereof, 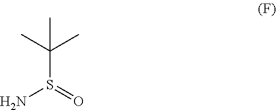
under conditions sufficient to provide a compound of Formula (G) or a stereoisomer thereof, or a salt thereof, 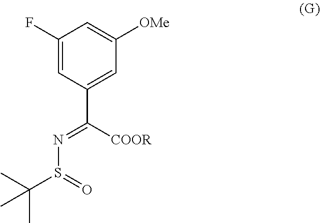
wherein R is C₁₋₅ alkyl;
(ii) contacting the compound of Formula (G) or a stereoisomer thereof, or a salt thereof, with a reducing agent to provide a compound of Formula (G-2), or a salt thereof, 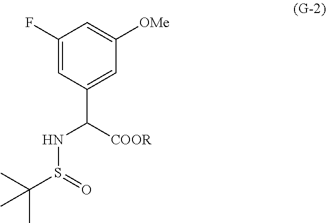
wherein R is C₁₋₅ alkyl;
(iii) contacting the compound of Formula (G-2) or a stereoisomer thereof, or a salt thereof, with a reducing agent to provide a compound of Formula (H) or a stereoisomer thereof, or a salt thereof, 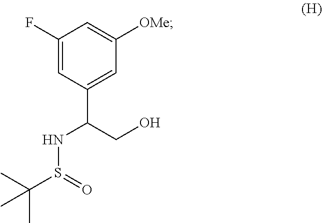 [SMILES: COc1cc(F)cc(C(CO)NS(=O)C(C)(C)C)c1]
and
iv) contacting the compound of Formula (H) or a stereoisomer thereof, or a salt thereof, with an acid to provide the compound of Formula (J) or a stereoisomer thereof, or a salt thereof.

12. The process of claim 11, wherein
the compound of Formula (F) has the structure of Formula (F-1): 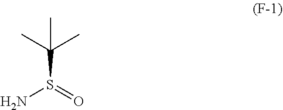
or a salt thereof, and
the compound of Formula (G) has the structure of Formula (G-1): 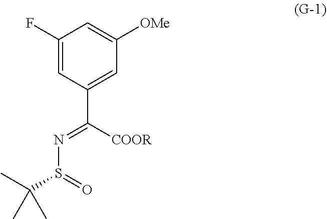
or a salt thereof;
wherein R is C₁₋₅ alkyl;
the compound of Formula (G-2) has the structure of Formula (G-3) 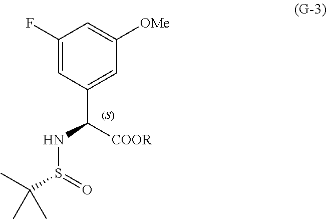
or a salt thereof,
wherein R is C₁₋₅ alkyl;
the compound of Formula (H) has the structure of Formula (H-1): 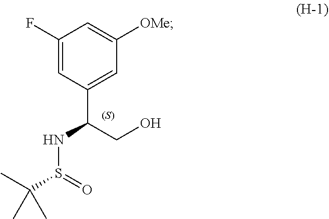
and
the compound of Formula (J) has the structure of Formula (J-1): 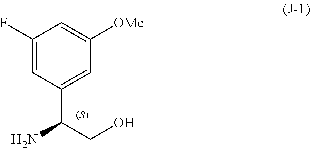
or a salt thereof.

13. The process of any one of claims 9-12, wherein the compound of Formula (J) has the structure of Formula (J-2):

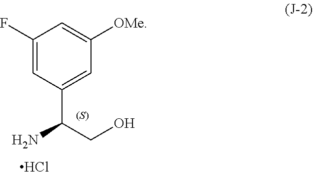

14. The process of any one of claims 9-13, wherein step (i) is conducted in the presence of a Lewis acid.

15. The process of claim 14, wherein the Lewis acid is MgSO₄, CuSO₄, Cs₂CO₃, Yb(OTf)₃, ZnCl₂, tris-(2,2,2-trifluoro ethyl)borate, trialkyl borates, diazabicycloundecene (DBU), KO^{t}Bu, TiCl₄, BF₃.OEt₂, Sc(OTf)₃ or a titanium alkoxide of Formula (K): 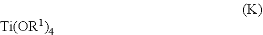
wherein R¹ is C₁₋₅ alkyl.

16. The process of claim 15, wherein the Lewis acid is Ti(OiPr)₄, or Ti(OEt)₄.

17. The process of any one of claims 9-10 and 13-16, wherein the reducing agent in step (ii) is borane, NaBH₄/BF₃.OEt₂, Sodium bis(2-methoxyethoxy)aluminum hydride, diisobutylaluminium hydride (DIBAL), or NaBH₄/I₂.

18. The process of any one of claims 9-10 and 13-16, wherein the reducing agent in step (ii) is borane.

19. The process of any one of claims 11-16, wherein the reducing agent in step (ii) is LiBH₄, NaBH₄, or ZnBH₄.

20. The process of any one of claims 11-16, wherein the reducing agent in step (iii) is borane, NaBH₄/BF₃.OEt₂, Sodium bis(2-methoxyethoxy)aluminum hydride, diisobutylaluminium hydride (DIBAL), or NaBH₄/I₂.

21. A compound of Formula (G-1) or a salt thereof 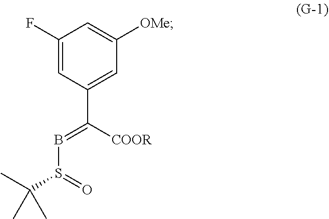
wherein R is C₁₋₅ alkyl.

22. A compound of Formula (G-3) or a salt thereof 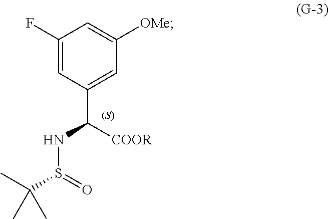
wherein R is C₁₋₅ alkyl.

23. A compound of Formula (H-1), or a salt thereof,

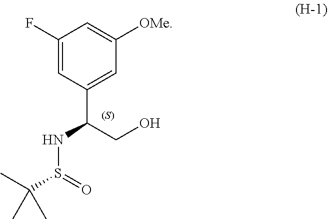

24. A compound of Formula (J-3)

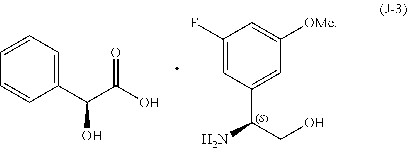

25. A compound of Formula (J-4)

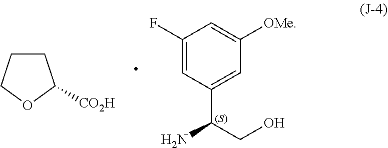

26. A compound of Formula (M-2), or a salt thereof,

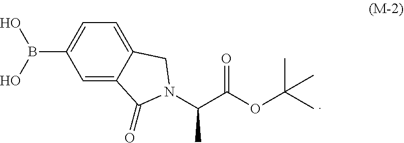 [SMILES: C[C@H](C(=O)OC(C)(C)C)N1Cc2ccc(B(O)O)cc2C1=O]

27. A compound of Formula (S), or a salt thereof, 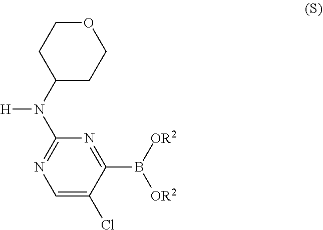
wherein R² and R³ are independently H, C₁₋₅ alkyl, or R² and R³ together with the atoms to which they are attached form a 5-or 6-membered ring optionally substituted with 1, 2, 3, or 4 C₁₋₃ alkyl.

28. A process for preparing a compound of Formula (N), or a salt thereof, 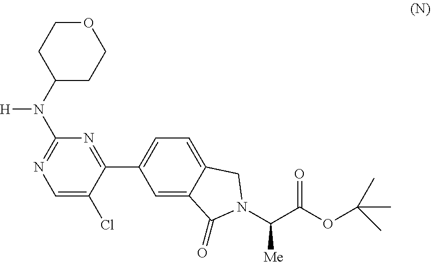 [SMILES: C[C@H](C(=O)OC(C)(C)C)N1Cc2ccc(-c3nc(NC4CCOCC4)ncc3Cl)cc2C1=O]
comprising:
contacting a compound of Formula (D), or a salt thereof, 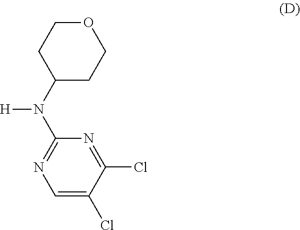 [SMILES: Clc1cnc(NC2CCOCC2)nc1Cl]
with a compound of Formula (M) 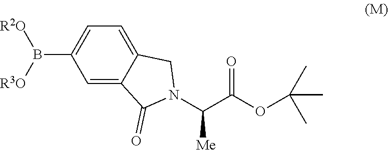
wherein R² and R³ are independently H, C₁₋₅ alkyl, or R² and R³ together with the atoms to which they are attached form a 5-or 6-membered ring optionally substituted with 1, 2, 3, or 4 C₁₋₃ alkyl;
under conditions sufficient to provide a compound of Formula (N), or a salt thereof.

29. The process of claim 28, wherein the process is conducted in the presence of an aqueous base and a palladium catalyst.

30. The process of claim 29, wherein the aqueous base is aqueous K₂CO₃, aqueous Na₂CO₃, aqueous Cs₂CO₃, aqueous LiOH, or aqueous K₃PO₄.

31. The process of claim 29, wherein the palladium catalyst is Pd(dppf)Cl₂, or Pd(OAc)₂ with a ligand selected from PPh₃, P(o-Tol)₃, PCy₃HBF₄, Dppf, Dppe, Xantphos, Xphos, BINAP (racemic, R, or S) and t-BuXphos.

32. The process of claim 29, wherein the palladium catalyst is Pd(dppf)Cl₂.

33. The process of any one of claims 28-32, wherein the compound of Formula (D) is prepared by a process comprising:
(i) contacting a compound of Formula (A), or a salt thereof, 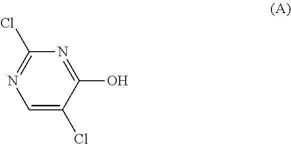
with a compound of Formula (B), or a salt thereof, 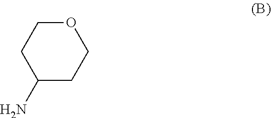
under conditions sufficient to provide a compound of Formula (C), or a salt thereof, 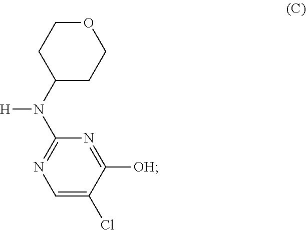 [SMILES: Oc1nc(NC2CCOCC2)ncc1Cl]
and
(ii) chlorinating the compound of Formula (C), or a salt thereof, to provide the compound of Formula (D), or a salt thereof.

34. The process of claim 33, wherein the compound of Formula (A) is a compound of Formula (A-1):

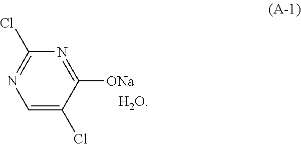

35. The process of any one of claims 28-34, further comprising
(iv) removing the tert-butyl group in the compound of Formula (N), or a salt thereof, to provide a compound of Formula (O), or a salt thereof, 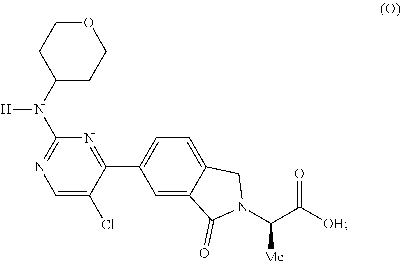 [SMILES: C[C@H](C(=O)O)N1Cc2ccc(-c3nc(NC4CCOCC4)ncc3Cl)cc2C1=O]
and
(v) coupling the compound of Formula (O) with a compound of Formula (J-1), or a salt thereof, 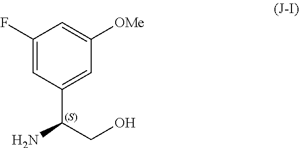
to provide a compound of Formula (I), or a pharmaceutically acceptable salt, solvate, or hydrate thereof,

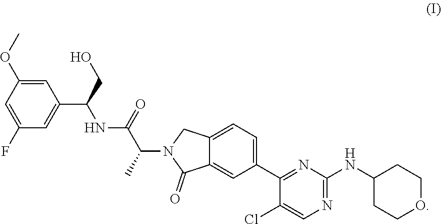 [SMILES: COc1cc(F)cc([C@@H](CO)NC(=O)[C@@H](C)N2Cc3ccc(-c4nc(NC5CCOCC5)ncc4Cl)cc3C2=O)c1]

36. The process of claim 35, wherein step (v) provides a monohydrate of a compound of Formula (I).

37. The process of claim 35, wherein the compound of Formula (J-1), or a salt thereof, is prepared by a process comprising:
(i) contacting a compound of Formula (E) 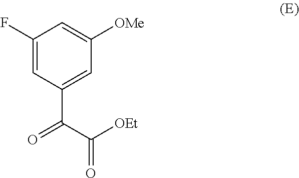 [SMILES: CCOC(=O)C(=O)c1cc(F)cc(OC)c1]
with a compound of Formula (F-1), or a salt thereof, 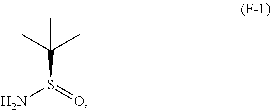
under conditions sufficient to provide a compound of Formula (G-1) 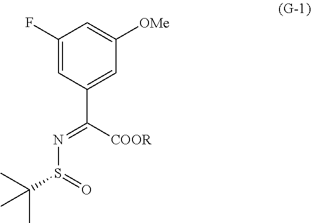
wherein R is C₁₋₅ alkyl;
(ii) contacting the compound of Formula (G-1) with a reducing agent to provide a compound of Formula (H-1), or a salt thereof, 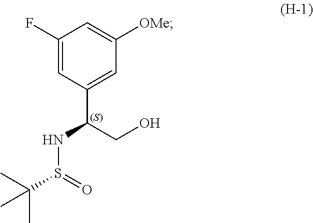
and
(iii) contacting the compound of Formula (H-1) with an acid to provide the compound of Formula (J-1), or a salt thereof.

38. The process of claim 35, wherein the compound of Formula (J-1), or a salt thereof, is prepared by a process comprising:
(i) contacting a compound of Formula (E) 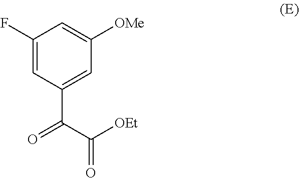 [SMILES: CCOC(=O)C(=O)c1cc(F)cc(OC)c1]
with a compound of Formula (F-1), or a salt thereof, 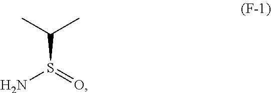
under conditions sufficient to provide a compound of Formula (G-1) 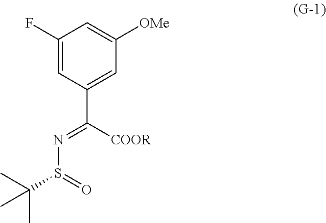
wherein R is C₁₋₅ alkyl;
(ii) contacting the compound of Formula (G-1) with a reducing agent to provide a compound of Formula (G-3), or a salt thereof 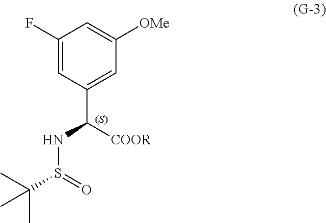
(iii) contacting the compound of Formula (G-3) with a reducing agent to provide a compound of Formula (H-1), or a salt thereof, 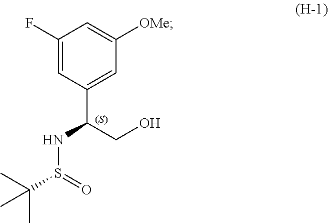
and
(iv) contacting the compound of Formula (H-1) with an acid to provide the compound of Formula (J-1), or a salt thereof.

39. A process for preparing a compound of Formula (I), or a pharmaceutically acceptable salt, solvate, or hydrate thereof, 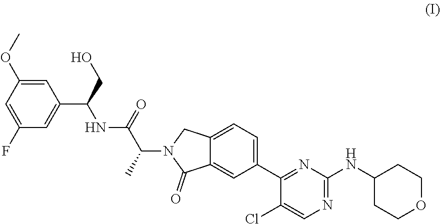 [SMILES: COc1cc(F)cc([C@@H](CO)NC(=O)[C@@H](C)N2Cc3ccc(-c4nc(NC5CCOCC5)ncc4Cl)cc3C2=O)c1]
comprising:
(i) contacting a compound of Formula (A-1), 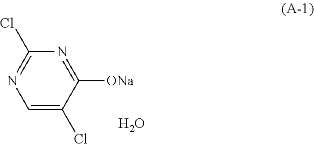
with a compound of Formula (B), or a salt thereof, 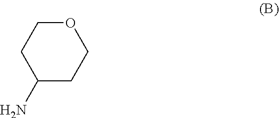
under conditions sufficient to provide a compound of Formula (C), or a salt thereof, 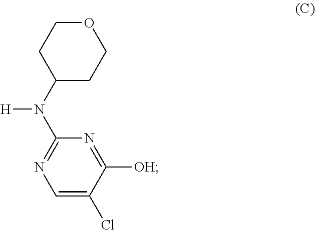 [SMILES: Oc1nc(NC2CCOCC2)ncc1Cl]
(ii) chlorinating the compound of Formula (C), or a salt thereof, to provide a compound of Formula (D), or a salt thereof, 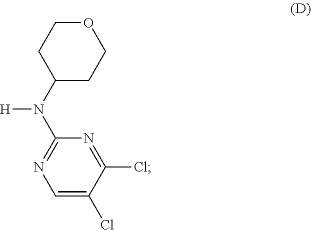 [SMILES: Clc1cnc(NC2CCOCC2)nc1Cl]
(iii) contacting the compound of Formula (D), or a salt thereof, with a compound of Formula (M) 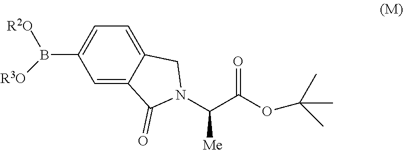
wherein R² and R³ are independently H, C₁₋₅ alkyl, or R² and R³ together with the atoms to which they are attached form a 5-or 6-membered ring optionally substituted with 1, 2, 3, or 4 C₁₋₃ alkyl;
under conditions sufficient to provide a compound of Formula (N), or a salt thereof, 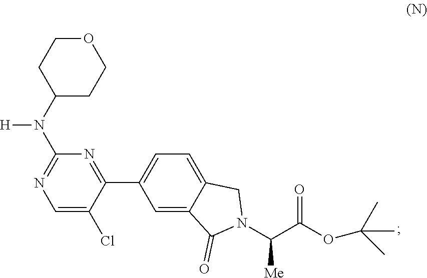 [SMILES: C[C@H](C(=O)OC(C)(C)C)N1Cc2ccc(-c3nc(NC4CCOCC4)ncc3Cl)cc2C1=O]
(iv) removing the tert-butyl group in the compound of Formula (N), or a salt thereof, to provide a compound of Formula (O), or a salt thereof, 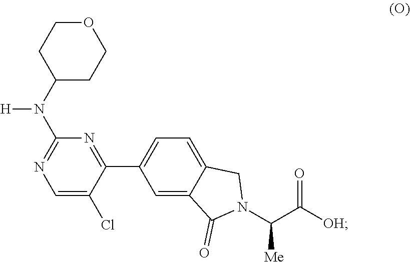 [SMILES: C[C@H](C(=O)O)N1Cc2ccc(-c3nc(NC4CCOCC4)ncc3Cl)cc2C1=O]
and
(v) coupling the compound of Formula (O) with a compound of Formula (J-2) 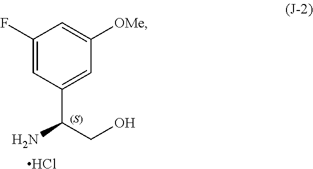
to provide the compound of Formula (I), or a pharmaceutically acceptable salt, solvate, or hydrate thereof;
wherein the compound of Formula (J-2) is prepared by a process comprising:
(v-i) contacting a compound of Formula (E) 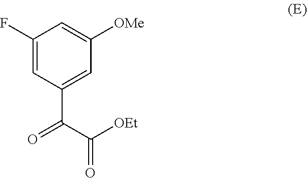 [SMILES: CCOC(=O)C(=O)c1cc(F)cc(OC)c1]
with a compound of Formula (F-1), or a salt thereof, 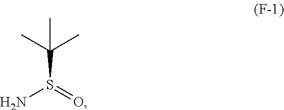
under conditions sufficient to provide a compound of Formula (G-1) 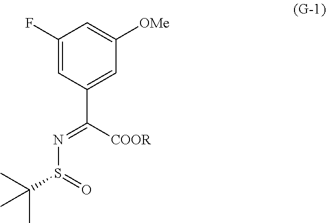
wherein R is C₁₋₅ alkyl;
(v-ii) contacting the compound of Formula (G-1) with a reducing agent to provide a compound of Formula (H-1), or a salt thereof, 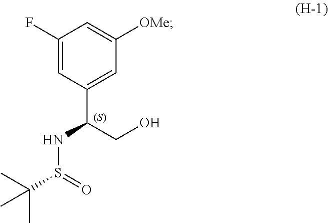
and
(v-iii) contacting the compound of Formula (H-1), or a salt thereof, with an acid to provide the compound of Formula (J-2).

40. A process for preparing a compound of Formula (I), or a pharmaceutically acceptable salt, solvate, or hydrate thereof, 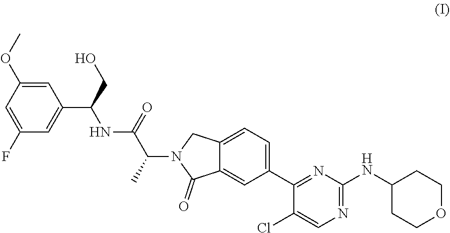 [SMILES: COc1cc(F)cc([C@@H](CO)NC(=O)[C@@H](C)N2Cc3ccc(-c4nc(NC5CCOCC5)ncc4Cl)cc3C2=O)c1]
comprising:
(i) contacting a compound of Formula (A-1), 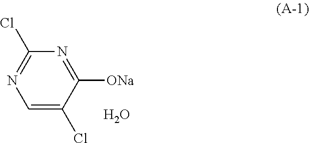
with a compound of Formula (B), or a salt thereof, 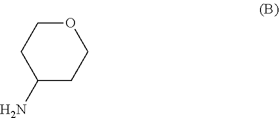
under conditions sufficient to provide a compound of Formula (C), or a salt thereof, 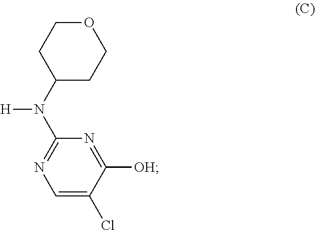 [SMILES: Oc1nc(NC2CCOCC2)ncc1Cl]
(ii) chlorinating the compound of Formula (C), or a salt thereof, to provide a compound of Formula (D), or a salt thereof, 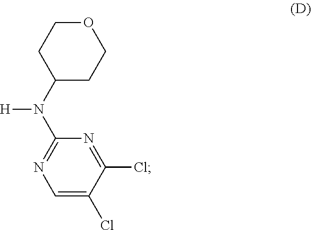 [SMILES: Clc1cnc(NC2CCOCC2)nc1Cl]
(iii) contacting the compound of Formula (D), or a salt thereof, with a compound of Formula (M) 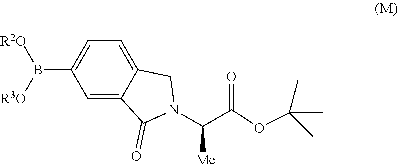
wherein R² and R³ are independently H, C₁₋₅ alkyl, or R² and R³ together with the atoms to which they are attached form a 5-or 6-membered ring optionally substituted with 1, 2, 3, or 4 C₁₋₃ alkyl;
under conditions sufficient to provide a compound of Formula (N), or a salt thereof, 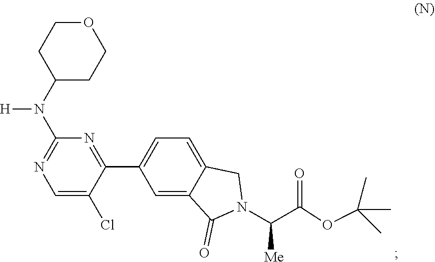 [SMILES: C[C@H](C(=O)OC(C)(C)C)N1Cc2ccc(-c3nc(NC4CCOCC4)ncc3Cl)cc2C1=O]
(iv) removing the tert-butyl group in the compound of Formula (N), or a salt thereof, to provide a compound of Formula (O), or a salt thereof, 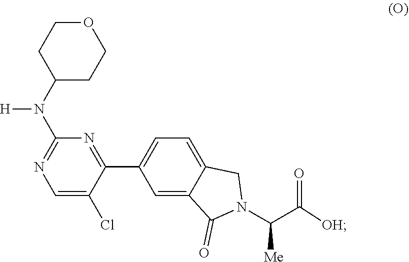 [SMILES: C[C@H](C(=O)O)N1Cc2ccc(-c3nc(NC4CCOCC4)ncc3Cl)cc2C1=O]
and
(v) coupling the compound of Formula (O) with a compound of Formula (J-2) 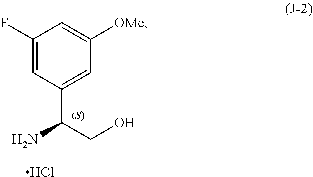
to provide the compound of Formula (I), or a pharmaceutically acceptable salt, solvate, or hydrate thereof;
wherein the compound of Formula (J-2) is prepared by a process comprising:
(v-i) contacting a compound of Formula (E) 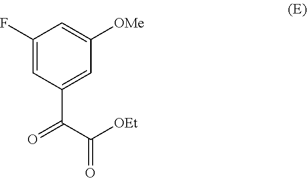 [SMILES: CCOC(=O)C(=O)c1cc(F)cc(OC)c1]
with a compound of Formula (F-1), or a salt thereof, 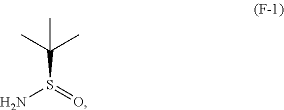
under conditions sufficient to provide a compound of Formula (G-1) 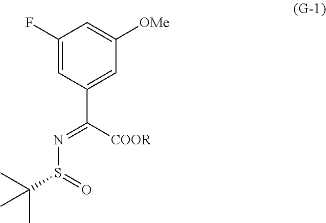
wherein R is C₁₋₅ alkyl;
(v-ii) contacting the compound of Formula (G-1) with a reducing agent to provide a compound of Formula (G-3), or a salt thereof, 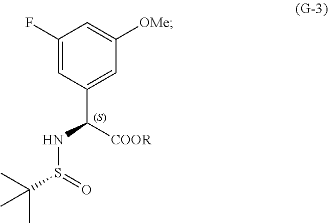
(v-iii) contacting the compound of Formula (G-3) with a reducing agent to provide a compound of Formula (H-1), or a salt thereof, 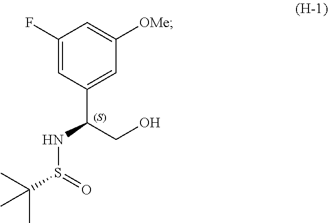
and
(v-iv) contacting the compound of Formula (H-1), or a salt thereof, with an acid to provide the compound of Formula (J-2).

41. The process of any one of claims 39-40, wherein step (v) provides a monohydrate of a compound of Formula (I).

42. A process for preparing a compound of Formula (L), or a salt thereof, 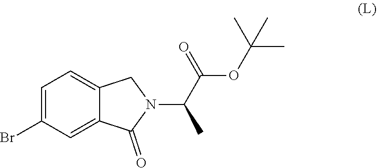 [SMILES: C[C@H](C(=O)OC(C)(C)C)N1Cc2ccc(Br)cc2C1=O]
comprising:
contacting a compound of Formula (P) 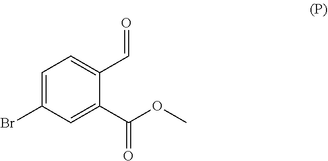
with a compound of Formula (Q), or a salt thereof, 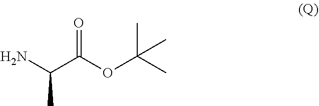
under conditions sufficient to provide the compound of Formula (L), or a salt thereof.

43. The process of claim 42, wherein the process is conducted in the presence of sodium triacetoxy borohydride (STAB), an amine, and a protic solvent.

44. A process for preparing a compound of Formula (N), or a salt thereof, 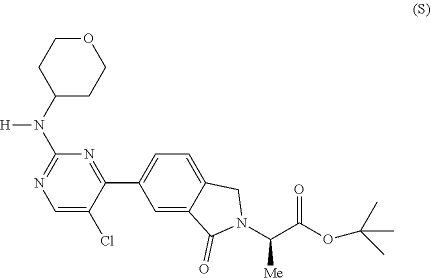 [SMILES: C[C@H](C(=O)OC(C)(C)C)N1Cc2ccc(-c3nc(NC4CCOCC4)ncc3Cl)cc2C1=O]
comprising:
contacting a compound of Formula (L) 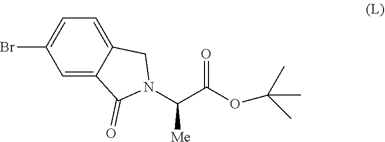 [SMILES: C[C@H](C(=O)OC(C)(C)C)N1Cc2ccc(Br)cc2C1=O]
with a compound of Formula (S) 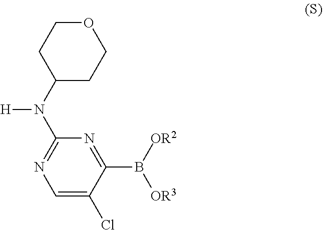
wherein R² and R³ are independently H, C₁₋₅ alkyl, or R² and R³ together with the atoms to which they are attached form a 5-or 6-membered ring optionally substituted with 1, 2, 3, or 4 C₁₋₃ alkyl;
under conditions sufficient to provide the compound of Formula (N).

45. A process for preparing a compound of Formula (I), or a pharmaceutically acceptable salt, solvate, or hydrate thereof, 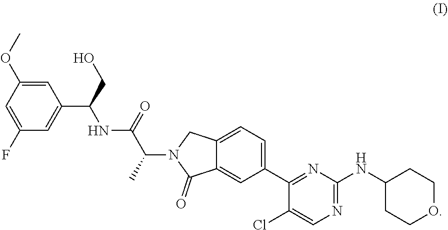 [SMILES: COc1cc(F)cc([C@@H](CO)NC(=O)[C@@H](C)N2Cc3ccc(-c4nc(NC5CCOCC5)ncc4Cl)cc3C2=O)c1]
comprising
contacting a compound of Formula (T), or a salt thereof, 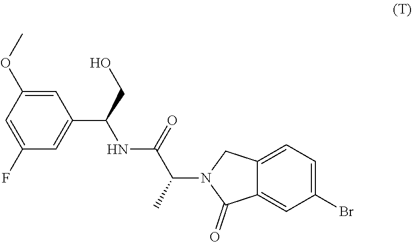 [SMILES: COc1cc(F)cc([C@@H](CO)NC(=O)[C@@H](C)N2Cc3ccc(Br)cc3C2=O)c1]
with a compound of Formula (S), or a salt thereof, 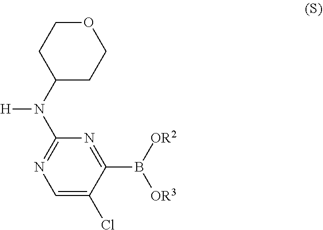
wherein R² and R³ are independently H, C₁₋₅ alkyl, or R² and R³ together with the atoms to which they are attached form a 5-or 6-membered ring optionally substituted with 1, 2, 3, or 4 C₁₋₃ alkyl;
under conditions sufficient to provide the compound of Formula (I), or a pharmaceutically acceptable salt, solvate, or hydrate thereof.

46. The process of claim 45, the compound of Formula (I) is a monohydrate.

47. A process for preparing a compound of Formula (I), or a pharmaceutically acceptable salt, solvate, or hydrate thereof, 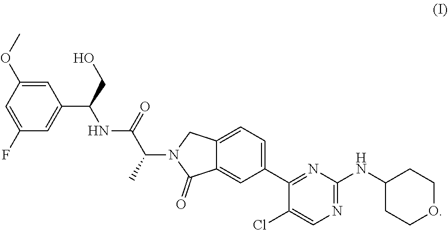 [SMILES: COc1cc(F)cc([C@@H](CO)NC(=O)[C@@H](C)N2Cc3ccc(-c4nc(NC5CCOCC5)ncc4Cl)cc3C2=O)c1]
comprising
contacting a compound of Formula (V), or a salt thereof, 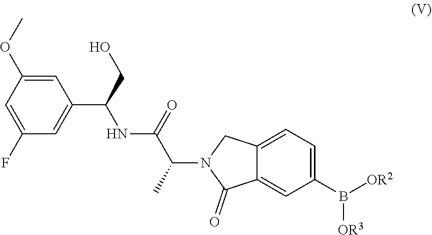
wherein R² and R³ are independently H, C₁₋₅ alkyl, or R² and R³ together with the atoms to which they are attached form a 5-or 6-membered ring optionally substituted with 1, 2, 3, or 4 C₁₋₃ alkyl;
with a compound of Formula (D), or a salt thereof, 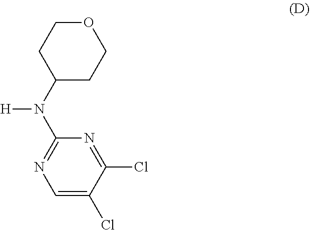 [SMILES: Clc1cnc(NC2CCOCC2)nc1Cl]
under conditions sufficient to provide the compound of Formula (I), or a pharmaceutically acceptable salt, solvate, or hydrate thereof.

48. The process of claim 45, wherein the compound of Formula (I) is a monohydrate.

49. The compound of Formula (I) prepared according to the process of any one of claim 39, 40, 45, or 47.

50. The compound of Formula (I), or a monohydrate thereof, prepared according to the process of any one of claim 39, 40, 45, or 47.

51. A composition comprising a compound of Formula (I) 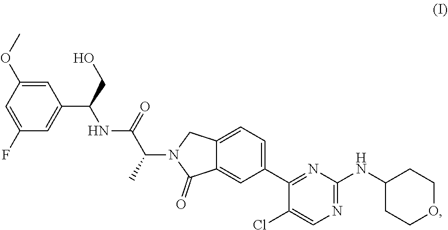 [SMILES: COc1cc(F)cc([C@@H](CO)NC(=O)[C@@H](C)N2Cc3ccc(-c4nc(NC5CCOCC5)ncc4Cl)cc3C2=O)c1]
wherein the composition comprises no more than 0.5% area/area of compounds of Formula (Z-1) and/or Formula (Z-2)

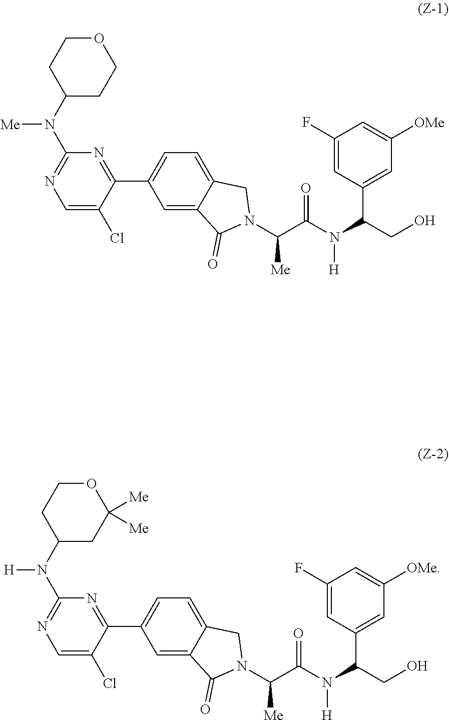

52. A process for preparing a compound of Formula (AB) 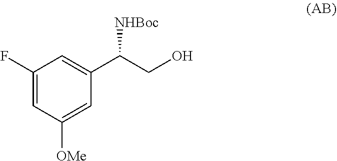 [SMILES: COc1cc(F)cc([C@@H](CO)NC(=O)OC(C)(C)C)c1]
comprising:
(i) contacting a compound of Formula (AA), or a salt thereof, 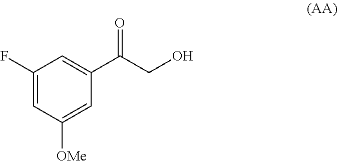 [SMILES: COc1cc(F)cc(C(=O)CO)c1]
with a transaminase to provide a compound of Formula (J-1), or a salt thereof, 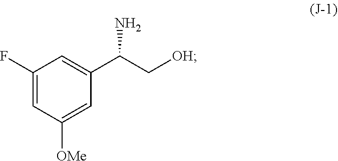 [SMILES: COc1cc(F)cc([C@H](N)CO)c1]
and
(ii) protecting the compound of Formula (J-1) to provide the compound of Formula (AB), wherein Boc is tert-butyloxycarbonyl.

53. The method of claim 52, further comprising deprotecting the compound of Formula (AB) to provide the compound of Formula (J-1), or a salt thereof:

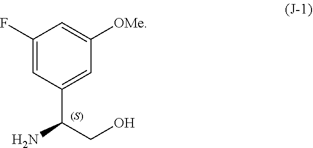

54. The method of claim 53, wherein the deprotecting is conducted in the presence of hydrochloric acid, trifluoro acetic acid, phosphoric acid, sulfuric acid, zinc bromide, catalytic iodine, acetyl chloride in methanol, or oxalyl chloride in methanol.

55. The method of claim 52, wherein the deprotecting is conducted in the presence of hydrochloric acid and the compound of Formula (J-1) is a compound of Formula (J-2)

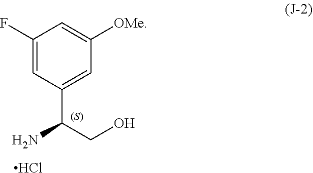

56. The method of claim 52, wherein the compound of Formula (AA) is prepared by contacting a compound of Formula (AC), or a salt thereof, 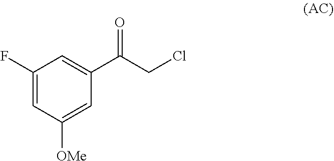 [SMILES: COc1cc(F)cc(C(=O)CCl)c1]
with alkali earth metal salts of carboxylic acids, or carboxylic acids, or mixtures thereof, in the presence of a solvent and water.

57. The method of claim 56, wherein the solvent is an alcohol, tetrahydrofuran, dimethylformamide, dimethylsulfoxide, acetonitrile, or a mixture thereof.

58. The method of claim 56, wherein the compound of Formula (AC) is prepared by contacting a compound of Formula (AD) 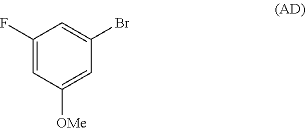
with a compound of Formula (AE), or a salt thereof, 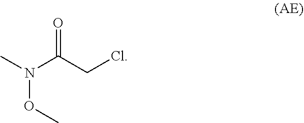
under conditions sufficient to provide the compound of Formula (AC).

59. A process for preparing a compound of Formula (J-2) 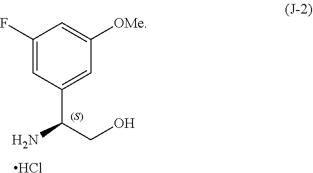
comprising:
(i) contacting a compound of Formula (AD) 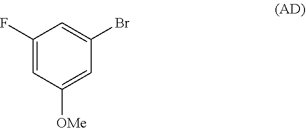
with magnesium metal and diethyl oxalate to provide a compound of Formula (E) 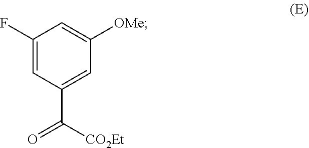 [SMILES: CCOC(=O)C(=O)c1cc(F)cc(OC)c1]
(ii) hydrolyzing the compound of Formula (E) to obtain a compound of Formula (AF), or a salt thereof, 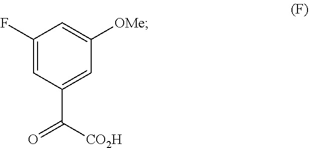 [SMILES: COc1cc(F)cc(C(=O)C(=O)O)c1]
(iii) contacting the compound of Formula (AF), or a salt thereof, with a transaminase to provide a compound of Formula (AG), or a salt thereof, 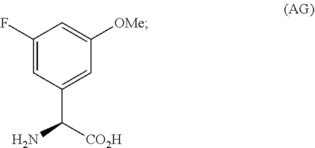 [SMILES: COc1cc(F)cc([C@H](N)C(=O)O)c1]
and
(iv) contacting the compound of Formula (AG) with a reducing agent and quenching the reaction with hydrochloric acid to provide the compound of Formula (J-2).

60. A compound of Formula (AH): 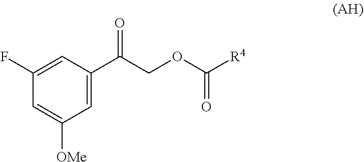
wherein R⁴ is H, C₂₋₆ alkyl or aryl.

61. A compound of Formula (AB), or a salt thereof 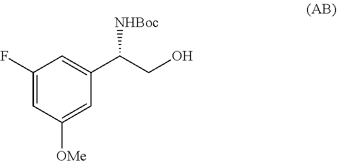 [SMILES: COc1cc(F)cc([C@@H](CO)NC(=O)OC(C)(C)C)c1]
wherein Boc is butyloxycarbonyl.
